(19) United States
(12) Patent Application Publication
(10) Pub. No.: US 2015/0099721 A1
(43) Pub. Date: Apr. 9, 2015

(54) TREATING CANCER WITH HSP90 INHIBITORY COMPOUNDS

(71) Applicant: Synta Pharmaceuticals Corp., Lexington, MA (US)

(72) Inventors: Jaime Acquaviva, Ashland, MA (US); Suqin He, West Roxbury, MA (US); David Proia, Newton, MA (US)

(73) Assignee: Synta Pharmaceuticals Corp., Lexington, MA (US)

(21) Appl. No.: 14/399,839

(22) PCT Filed: May 10, 2013
(86) PCT No.: PCT/US2013/040565

§ 371 Date: Nov. 7, 2014

Related U.S. Application Data
(60) Provisional application No. 61/651,623, filed on May 25, 2012.
(60) Provisional application No. 61/645,197, filed on May 10, 2012.

(51) Int. Cl. C07D 403/04; A61K 45/06; A61K 31/4545; A61K 31/4196; C07F 9/6518

(52) U.S. Cl. CPC C07D 403/04; A61K 31/4196; C07F 9/65185; A61K 31/4545; A61K 45/06
USPC 514/80; 514/384; 514/383; 514/318

(57) ABSTRACT

A method for treating cancer with a mutation in ROS or RET, comprising identifying a subject with a cancer with a mutation in ROS or RET, and administering to the subject an effective amount of a compound according to formulae (I) OR (la) or a tautomer, or a pharmaceutically acceptable salt thereof, wherein the variables structural formulae are defined herein.

### CROSS-REFERENCE TO RELATED PATENTS

This application claims the benefit of priority to U.S. Provisional Patent Application Nos. 61/645,197, filed on May 10, 2012, and 61/651,623, filed on May 25, 2012. The contents of each of these applications are incorporated herein by reference.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It has been found that Hsp90 inhibitors, such as certain triazolone Hsp90 inhibitors described herein, are particularly effective in treating cancer harboring a mutation in ROS protein, particularly in treating non-small cell lung cancer (NSCLC) or glioblastoma harboring a mutation in ROS such as v-ROS, Mcf3, FIG-ROS, SLC34A2-ROS, or CD74-ROS fusions. It is also found that Hsp90 inhibitors such as certain triazolone Hsp90 inhibitory compounds are particularly effective in treating cancer harboring a mutation in ROS protein wherein the cancer has previously been treated with an anticancer agent and is no longer responsive to the treatment.

The method described herein includes utilizing Hsp90 inhibitors according to formulae (I) or (Ia), or a compound in Tables 1 or 2, for the treatment of cancer harboring a mutation in ROS protein in a subject in need thereof. The method of treating a subject with cancer includes the steps of identifying the presence of a mutation in ROS protein in a sample from the subject, and administering to the subject an effective amount of an Hsp90 inhibitor according to formulae (I) or (Ia) or a compound in Tables 1 or 2. For example, the sample from the subject can be assessed for the presence of a mutation in ROS protein. If the sample harbors the ROS mutation, the subject is administered an effective amount of an Hsp90 inhibitor according to formulae (I) or (Ia) or a compound in Tables 1 or 2; and if the sample does not harbor the ROS mutation, the subject is preferably administered an anti-cancer therapy other than an Hsp90 inhibitor according to formulae (I) or (Ia) or a compound in Tables 1 or 2. In one embodiment, the Hsp90 inhibitor is ganetespib. In one embodiment, ganetespib is administered as a single agent. In another embodiment, ganetespib is administered in combination with one or more additional therapeutic agents. In one embodiment, the one or more additional therapeutic agents are BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline. In one embodiment, the Hsp90 inhibitor is ganetespib and the additional anticancer agent is crizotinib.

In one embodiment, the cancer harboring a mutation in ROS protein is non-small cell lung cancer. In one embodiment, the non-small cell lung cancer has a v-ROS fusion. In one embodiment, the non-small cell lung cancer has an Mcf3 fusion. In one embodiment, the non-small cell lung cancer has a FIG-ROS fusion. In one embodiment, the non-small cell lung cancer has an SLC34A2-ROS fusion. In one embodiment, the non-small cell lung cancer has a CD74-ROS fusion. In one embodiment, the cancer harboring an alteration, mutation or rearrangement in a ROS gene or gene product is glioblastoma. In one embodiment, the cancer harboring a mutation in ROS protein is brain, lung, stomach, breast, liver, colon, kidney, or head and neck cancer.

In one embodiment, the method also includes treating cancer, wherein the cancer has been previously treated with an anticancer agent and is no longer responsive to the treatment. The method includes the steps of identifying a subject wherein the subject has previously been treated with an anticancer agent and is no longer responsive to the earlier treatment and administering to the identified subject an effective amount of an Hsp90 compound according to formulae (I) or (Ia) or a compound in Tables 1 or 2. In one embodiment, the cancer subject has been previously treated with crizotinib and is no longer responsive to crizotinib treatment, and the subject is then treated with ganetespib. In one embodiment, the subject has NSCLC and has been previously treated with crizotinib and is no longer responsive to the treatment, and the subject is then treated with ganetespib.

In one embodiment, the method of treating cancer harboring a mutation in ROS protein may include the administration of one or more therapeutic agents in addition to an Hsp90 compound according to formulae (I) or (Ia) or a compound in Tables 1 or 2. (As used in the above context, “a” or “an” is intended to mean “at least one.”)

The invention also provides the use of a compound of structural formula (I) or (Ia) or a compound in Tables 1 or 2 or a pharmaceutically acceptable salt thereof for the manufacture of a medicament for the treatment of cancer harboring a mutation in ROS protein. The invention further provides the use of a compound of structural formula (I) (Ia) or a compound in Tables 1 or 2 or a pharmaceutically acceptable salt thereof for the manufacture of a medicament for the treatment of cancer harboring a mutation in ROS protein in combination with BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline. In one embodiment, the medicament is ganetespib in combination with crizotinib.

### BRIEF DESCRIPTION OF THE FIGURES

FIG. 1A shows dose response curves for ganetespib and crizotinib in HCC78 cells exposed to the respective drug for 72 hr.

FIG. 1B shows a western blot analysis of ROS phosphorylation in HCC78 cells treated with ganetespib for 24 hr at 250 nM.

FIG. 1C shows the killing effects of HCC78 cells by ganetespib, crizotinib or the combination of the two drugs at the indicated concentrations.

FIG. 1D shows western blot analysis of ROS phosphorylation in CD74-ROS and FIG-ROS expressing Ba/F3 cells treated for 24 hr with ganetespib at a concentration of 100 nM.

FIG. 2A shows dose response curves for ganetespib in TPC-1 cells exposed to drug for 72 hr.

FIG. 2B shows western blot analysis of CCDC6-RET (total and phosphorylated) in TCP-1 cells treated with ganetespib for 24 hr at doses indicated.

FIG. 3 shows that ganetespib and crizotinib inhibited viability of different cancer cells driven by oncogenic ROS1 fusion.

FIG. 4 shows that ganetespib significantly inhibited the viability of more cancer cells driven by oncogenic ROS1 fusion as compared with crizotinib.

FIG. 5 shows that Hsp90 inhibition by ganetespib leads to degradation of ROS1 fusion protein.

FIG. 6 further shows Hsp90 inhibition by ganetespib leading to degradation of ROS1 fusion protein based on Western blot analysis of ROS phosphorylation in CD74-ROS and FIG-ROS expressing Ba/F3 cells treated for 24 hr with ganetespib (100 nM).

FIG. 7 shows that ganetespib significantly inhibited the viability of TPC-1 cancer cells driven by oncogenic RET fusion protein as compared with other agents.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in the compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocycle included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring may be a 5-8 membered monocyclic heteroaryl ring. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³³C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C1-C4 alkyl, C1-C4 haloalkyl, C1-C4 alkoxy, C1-C4 haloalkoxy, C1-C4 hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

Unless indicated otherwise, the compounds described herein containing reactive functional groups such as carboxy, hydroxy, thiol and amino moieties, also include corresponding protected derivatives thereof. “Protected derivatives” are those compounds in which a reactive site or sites are blocked with one or more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

As used herein, the term “compound(s) described herein” or similar terms refers to a compound of formulae (I), or (Ia) or a compound in Tables 1 or 2 or a tautomer or pharmaceutically acceptable salt thereof. Also included in the scope of the embodiments are a solvate, clathrate, hydrate, polymorph, prodrug, or protected derivative of a compound of formulae (I), or (Ia), or a compound in Tables 1 or 2.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound's enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water).

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I) or (Ia) or a compound in Tables 1 or 2 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can typically be prepared using well-known methods, such as those described by BURGER'S MEDICINAL CHEMISTRY AND DRUG DISCOVERY, (Manfred E. Wolff Ed., 5th ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

The human c-ROS gene was mapped to the human chromosome 6 region, 6q16-6q22. This region of chromosome 6 is involved in nonrandom chromosomal rearrangement in specific neoplasias, including acute lymphoplastic leukemia, malignant melanoma, and ovarian carcinomas. c-ROS gene up-regulation and/or mutation was found mainly in brain and lung cancers, in addition to chemically induced stomach cancer, breast fibroadenomas, liver, colon, and kidney cancers.

In a survey of 45 different human cell lines, ROS was found to be expressed in 56% of glioblastoma-derived cell lines at high levels (ranging from 10 to 60 transcripts per cell), while not expressed at all or expressed minimally in the remaining cell lines. Moreover, no expression of ROS gene was observed in normal brain tissues; thus, the high level of ROS expression in glioblastoma seems specific.

ROS kinase is a proto-oncogenic receptor tyrosine kinase whose expression is tightly restricted during development. It is normally expressed in adult murine and human epithelial cells of the epididymis. Transgenic mice lacking the c-ros gene are infertile. Ectopic expression of c-ROS has been reported in meningiomas and astrocytomas. ROS kinase is up-regulated in human glioma: 30% of malignant glioma tumors are ROS positive. An oncogenic fusion protein between PIST (aka FIG) and ROS, resulting from an intra-chromosomal homozygous deletion of 240 kilobases on 6q21, is found in glioblastoma multiform. PIST (aka FIG) is a peripheral membrane protein associated with the Golgi apparatus. Unlike other fusion RTK oncogenes, the mechanism of activation of PIST-ROS does not appear to be dimerization: the PIST-ROS fusion protein appears to be monomeric in vivo. Rather, activation of the fused ROS kinase appears to depend upon translocation to the Golgi apparatus: deletion of second coiled-coil region of PIST, crucial for Golgi localization, appears to eliminate the transformation capacity of PIST-ROS. c-ROS may also be activated epigenetically, suggesting caution when using 5-aza-dC for treating glioma.

As used herein, a “subject with a mutation” in ROS gene associated with cancer, or a “subject with a cancer with a mutation” in ROS gene associated with cancer, and the like, are understood as a subject having cancer, wherein the tumor has at least one alteration (e.g., 1, 2, 3, 4, 5, 6, 7, 8, 9, 10, or more) in the indicated gene from the wild-type sequence in the gene and/or transcriptional, translational, and/or splicing control regions of the gene that result in the cell becoming cancerous, e.g., developing characteristics such as uncontrolled proliferation, immortality, metastatic potential, rapid growth and proliferation rate, decreased cell death/apoptosis, and certain characteristic morphological features. Mutations include, for example, insertions, deletions, truncations, point mutations, and translocations. Mutations within a gene product can result in constituent activation of the gene product. Mutations that include alterations in transcriptional, translational, or splicing control regions can result in aberrant expression, typically over-expression, of a wild-type gene product. It is understood that not all gene mutations, even in oncogenes, result in a cell becoming cancerous. Mutations that result in oncogenesis are well known in the art. Methods to test mutations for oncogenic activity are well known in the art.

Rearranged during Transfection (RET) is a transmembrane tyrosine kinase expressed in central and peripheral nervous system and neural crest-derived cells. RET protein comprises an extracellular portion with four cadherin-like domains and a cysteine-rich region important for intermolecular interactions; a hydrophobic transmembrane domain; an intracellular part comprising the juxtamembrane domain with regulatory function and the catalytic domain that phosphorylates the tyrosine residues of substrates. RET is involved in the development of enteric nervous system and renal organogenesis during embryonic life. Mutations of RET are associated with a subset of colorectal cancer and are commonly found in hereditary and sporadic thyroid cancer. Activating point mutations in the cysteine-rich or the kinase domain of RET cause multiple endocrine neoplasia type 2 (MEN2), a group of familial cancer syndromes characterized by medullary thyroid carcinoma, pheochromocytoma, parathyroid hyperplasia and ganglioneuromatosis of the gastroenteric mucosa. Rearranged forms of RET are detected in the majority of papillary thyroid carcinomas (PTC). See, e.g., Curr Med Chem. 2011; 18(2): 162-75, and the references cited therein for more information and identification of mutations in RET.

A mutation can be detected using any of a number of known methods in the art. The specific method to detect the mutation will depend, for example, on the type of mutation to be detected. For example, alterations in nucleic acid sequences can be easily detected using polymerase chain reaction and fluorescence in situ hybridization methods (FISH). Protein expression levels can be detected, for example, using immunohistochemistry. An aberrant expression level of a wild-type protein can be used as a surrogate for detection of a mutation in a transcriptional, translational, and/or splicing control regions of the gene without direct detection of the specific genetic change in the nucleic acid in the subject sample. The specific method of detection of the mutation is not a limitation of the invention. Methods to compare protein expression levels to appropriate controls are well known in the art.

In a preferred embodiment, when multiple tests are used to detect a mutation and one is positive, the mutation is considered to be present. The methods do not require that multiple assays be performed to detect a mutation.

Mutations or protein expression levels are preferably detected in a subject sample from the cancer tissue or tumor tissue, e.g., cells, extracellular matrix, and other naturally occurring components associated with the tumor. The mutation or expression level can be detected in a biopsy sample or in a surgical sample after resection of the tumor. The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In an embodiment, the sample is removed from the subject. In a particular embodiment, the sample is urine or serum. In an embodiment, the sample comprises cells. In another embodiment, the sample does not comprise cells. In certain embodiments, the sample can be the portion of the subject that is imaged. Samples are typically removed from the subject prior to analysis, however, tumor samples can be analyzed in the subject, for example, using imaging or other detection methods.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that identifying a subject or selecting a subject as having one or more mutations in one or more genes of interest, having a wild-type gene, or having a change in the expression level of a protein, and can include any of a number of acts including, but not limited to, performing a test and observing a result that is indicative of a subject having a specific mutation; reviewing a test result of a subject and identifying the subject as having a specific mutation; reviewing documentation on a subject stating that the subject has a specific mutation and identifying the subject as the one discussed in the documentation by confirming the identity of the subject e.g., by an identification card, hospital bracelet, asking the subject for his/her name and/or other personal information to confirm the subjects identity.

As used herein, the term “refractory” cancer or tumor is understood as a malignancy which is either initially unresponsive to chemo- or radiation therapy, or which becomes unresponsive over time. A cancer refractory to on intervention may not be refractory to all interventions. A refractory cancer is typically not amenable to treatment with surgical interventions.

As used herein, “relapse” is understood as the return of a cancer or the signs and symptoms of a cancer after a period of improvement.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, “detecting”, “detection” and the like are understood that an assay performed for identification of a specific analyte in a sample, e.g., a gene or gene product with a mutation, or the expression level of a gene or gene product in a sample, typically as compared to an appropriate control cell or tissue. The specific method of detection used is not a limitation of the invention. The detection method will typically include comparison to an appropriate control sample.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of signal detected or protein expression in a control sample that consists of a group of measurements may be determined, e.g., based on any appropriate statistical measure, such as, for example, measures of central tendency including average, median, or modal values.

As used herein, the term “pharmaceutically acceptable salt” refers to a salt prepared from a compound of formulae (I) or (Ia) or a compound in Tables 1 or 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I) or (Ia) or a compound in Tables 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I) or (Ia) or a compound in Tables 1 or 2. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17th ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound described herein being used. In cases where no amount is expressly noted, an effective amount should be assumed. Non-limiting examples of an effective amount of a compound described herein are provided herein below. In a specific embodiment, the method includes treating, managing, or ameliorating a disease or disorder, e.g. a proliferative disorder, or one or more symptoms thereof, comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the one or more additional therapeutic agents are dosed on independent schedules. In another embodiment, the Hsp90 inhibitor and the one or more additional therapeutic agents are dosed on approximately the same schedule. In another embodiment, the Hsp90 inhibitor and the one or more additional therapeutic agents are dosed concurrently or sequentially on the same day. In another embodiment, the Hsp90 inhibitor and the one or more additional therapeutic agents are dosed sequentially on different days.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomerically or enantiomerically pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

In one aspect, the method includes treating a subject with cancer with a mutation in ROS, comprising the steps of identifying a subject with cancer with a mutation in ROS, and administering an effective amount of an Hsp90 inhibitory compound shown in Tables 1 or 2, or according to formula (I) or (Ia) as set forth below:

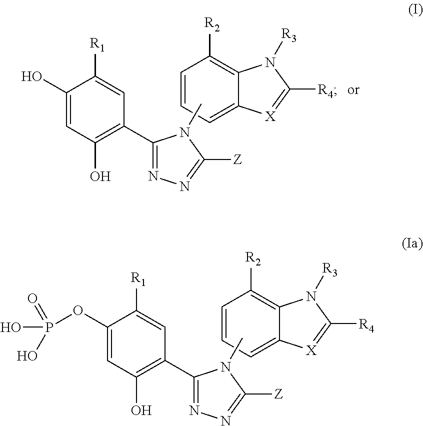

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)R₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)R₁₀R₁₁, —SC(O)NR₁₀R₁₁, —SC(NR₈)R₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)R₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)_{2;}
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)R₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘ(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ may be —H, lower alkyl, lower alkoxy, lower cycloalkyl, or lower cycloalkoxy.

In another embodiment, in formula (I) or (Ia), R₁ may be —H, methyl, ethyl, propyl, isopropyl, cyclopropyl, methoxy, ethoxy, propoxy, or cyclopropoxy.

In another embodiment, in formula (I) or (Ia), R₃ may be —H, a lower alkyl, a lower cycloalkyl, —C(O)N(R₂₇)₂, or —C(O)OH, wherein R₂₇ is —H or a lower alkyl.

In another embodiment, in formula (I) or (Ia), R₃ may be —H, methyl, ethyl, n-propyl, isopropyl, cyclopropyl, n-butyl, sec-butyl, tert-butyl, n-pentyl, n-hexyl, —C(O)OH, —(CH₂)ₘ(O)OH, —CH₂OCH₃, —CH₂CH₂OCH₃, or —C(O)N(CH₃)_{2.}

In one embodiment, R₄ may be H or a lower alkyl.

In another embodiment, in formula (I) or (Ia), R₄ may be —H, methyl, ethyl, propyl, isopropyl or cyclopropyl.

In another embodiment, in formula (I) or (Ia), R₁ may be —H, —OH, —SH, —NH₂, a lower alkoxy or a lower alkyl amino.

In another embodiment, in formula (I) or (Ia), R₁ may be —H, —OH, methoxy or ethoxy.

In another embodiment, in formula (I) or (Ia), Z is —OH.

In another embodiment, in formula (I) or (Ia), Z is —SH.

In another embodiment, in formula (I) or (Ia), R₂ is may be —H, —OH, —SH, —NH₂, a lower alkoxy or a lower alkyl amino.

In another embodiment, in formula (I) or (Ia), R₂ may be —H, —OH, methoxy, or ethoxy.

In another embodiment, in formula (I) or (Ia), R₁ is may be —H, methyl, ethyl, propyl, isopropyl, cyclopropyl, methoxy, ethoxy, propoxy, or cyclopropoxy; R₃ may be —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, or —C(O)N(CH₃)₂; R₄ may be —H, methyl, ethyl, propyl, isopropyl or cyclopropyl; R₂ may be —H, —OH, —SH, —NH₂, a lower alkoxy or a lower alkyl amino; and Z is OH.

In another embodiment, in formula (I) or (Ia), R₁ is may be —H, methyl, ethyl, propyl, isopropyl, cyclopropyl, methoxy, ethoxy, propoxy, or cyclopropoxy; R₃ may be —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, or —C(O)N(CH₃)₂; R₄ may be —H, methyl, ethyl, propyl, isopropyl or cyclopropyl; R₂ may be —H, —OH, —SH, —NH₂, a lower alkoxy or a lower alkyl amino; and Z is SH.

In another embodiment, the compound is selected from the group consisting of:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, the compound is selected from the group consisting of
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCl salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole,
3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

In another embodiment, the compound is selected from the group consisting of
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate, or a tautomer, or a pharmaceutically acceptable salt thereof.

Hsp90 inhibitory compounds, as well as tautomers or pharmaceutically acceptable salts thereof that may be used in the methods described herein are depicted in Tables 1 or 2.

TABLE 1

| | STRUCTURE | TAUTOMERIC STRUCTURE | NAME |
|---|---|---|---|
| 1 | 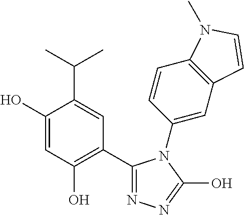 [SMILES: CC(C)c1cc(-c2nnc(O)n2-c2ccc3c(ccn3C)c2)c(O)cc1O] | 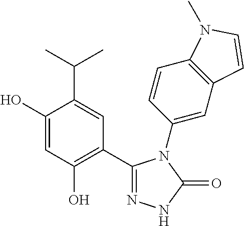 [SMILES: CC(C)c1cc(-c2n[nH]c(=O)n2-c2ccc3c(ccn3C)c2)c(O)cc1O] | 3-(2,4-DIHYDROXY-5- ISOPROPYL-PHENYL)-4-(1- METHYL-INDOL-5-YL)-5- HYDROXY-[1,2,4] TRIAZOLE (GANETESPIB) |
| 2 | 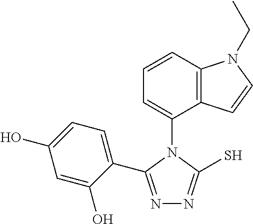 [SMILES: CCn1ccc2c(-n3c(S)nnc3-c3ccc(O)cc3O)cccc21] | 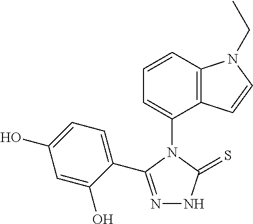 [SMILES: CCn1ccc2c(-n3c(-c4ccc(O)cc4O)n[nH]c3=S)cccc21] | 3-(2,4-DIHYDROXYPHENYL)-4- (1-ETHYL-INDOL-4-YL)-5- MERCAPTO-[1,2,4] TRIAZOLE |
| 3 | 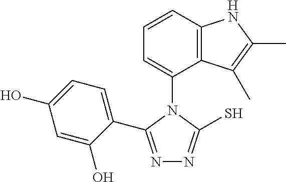 [SMILES: Cc1[nH]c2cccc(-n3c(S)nnc3-c3ccc(O)cc3O)c2c1C] | 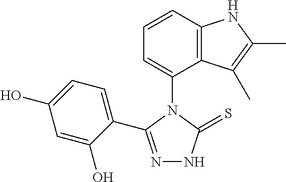 [SMILES: Cc1[nH]c2cccc(-n3c(-c4ccc(O)cc4O)n[nH]c3=S)c2c1C] | 3-(2,4-DIHYDROXY-PHENYL)-4- (2,3-DIMETHYL-1H-INDOL-4- YL)-5-MERCAPTO-[1,2,4] TRIAZOLE |
| 4 | 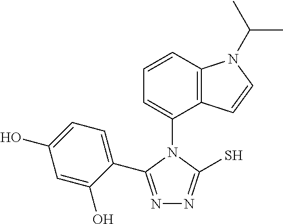 [SMILES: CC(C)n1ccc2c(-n3c(S)nnc3-c3ccc(O)cc3O)cccc21] | 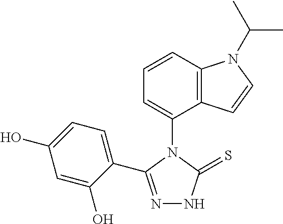 [SMILES: CC(C)n1ccc2c(-n3c(-c4ccc(O)cc4O)n[nH]c3=S)cccc21] | 3-(2,4-DIHYDROXYPHENYL)-4- (1-ISOPROPYL-INDOL-4-YL)-5- MERCAPTO-[1,2,4] TRIAZOLE |
| 5 | 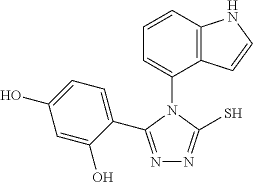 [SMILES: Oc1ccc(-c2nnc(S)n2-c2cccc3[nH]ccc23)c(O)c1] | 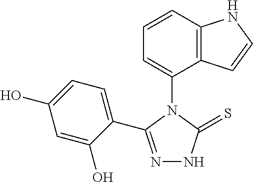 [SMILES: Oc1ccc(-c2n[nH]c(=S)n2-c2cccc3[nH]ccc23)c(O)c1] | 3-(2,4-DIHYDROXY-PHENYL)-4- (INDOL-4-YL)-5-MERCAPTO- [1,2,4] TRIAZOLE |
| 6 | 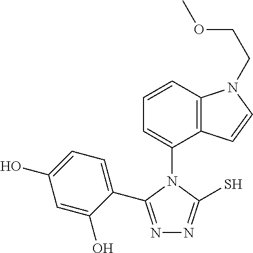 [SMILES: COCCn1ccc2c(-n3c(S)nnc3-c3ccc(O)cc3O)cccc21] | 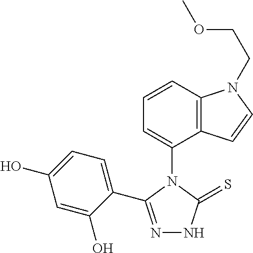 [SMILES: COCCn1ccc2c(-n3c(-c4ccc(O)cc4O)n[nH]c3=S)cccc21] | 3-(2,4-DIHYDROXY-PHENYL)-4- [1-(2-METHOXYETHOXY)- INDOL-4-YL]-5-MERCAPTO- [1,2,4] TRIAZOLE |
| 7 | 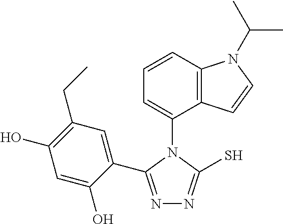 [SMILES: CCc1cc(-c2nnc(S)n2-c2cccc3c2ccn3C(C)C)c(O)cc1O] | 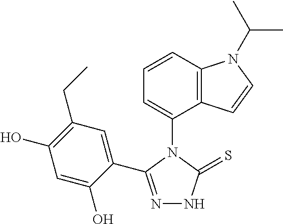 [SMILES: CCc1cc(-c2n[nH]c(=S)n2-c2cccc3c2ccn3C(C)C)c(O)cc1O] | 3-(2,4-DIHYDROXY-5-ETHYL- PHENYL)-4-(1-ISOPROPYL- INDOL-4-YL)-5-MERCAPTO- [1,2,4] TRIAZOLE |
| 8 | 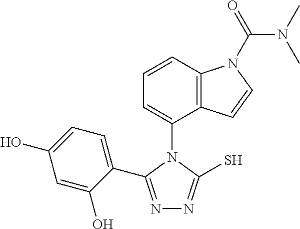 [SMILES: CN(C)C(=O)n1ccc2c(-n3c(S)nnc3-c3ccc(O)cc3O)cccc21] | 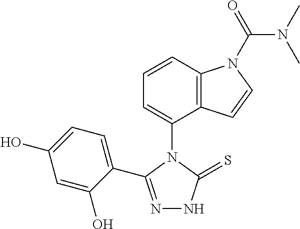 [SMILES: CN(C)C(=O)n1ccc2c(-n3c(-c4ccc(O)cc4O)n[nH]c3=S)cccc21] | 3-(2,4-DIHYDROXY-5-ETHYL- PHENYL)-4-[1-(DIMETHYL- CARBAMOYL)-INDOL-4-YL]-5- MERCAPTO-[1,2,4] TRIAZOLE |
| 9 | 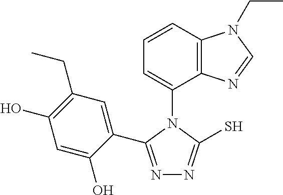 [SMILES: CCc1cc(-c2nnc(S)n2-c2cccc3c2ncn3CC)c(O)cc1O] | 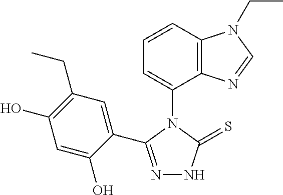 [SMILES: CCc1cc(-c2n[nH]c(=S)n2-c2cccc3c2ncn3CC)c(O)cc1O] | 3-(2,4-DIHYDROXY-5-ETHYL- PHENYL)-4-(1-ETHYL- BENZOIMIDAZOL-4-YL)-5- MERCAPTO-[1,2,4] TRIAZOLE |
| 10 | 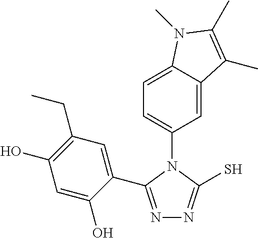 [SMILES: CCc1cc(-c2nnc(S)n2-c2ccc3c(c2)c(C)c(C)n3C)c(O)cc1O] | 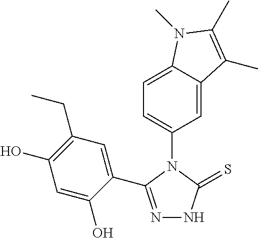 [SMILES: CCc1cc(-c2n[nH]c(=S)n2-c2ccc3c(c2)c(C)c(C)n3C)c(O)cc1O] | 3-(2,4-DIHYDROXY-5-ETHYL- PHENYL)-4-(1,2,3-TRIMETHYL- INDOL-5-YL)-5-MERCAPTO- [1,2,4] TRIAZOLE |
| 11 | 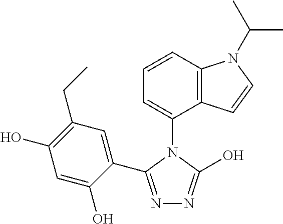 [SMILES: CCc1cc(-c2nnc(O)n2-c2cccc3c2ccn3C(C)C)c(O)cc1O] | 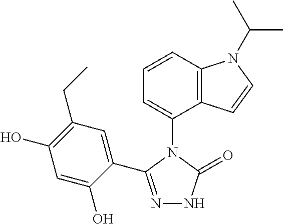 [SMILES: CCc1cc(-c2n[nH]c(=O)n2-c2cccc3c2ccn3C(C)C)c(O)cc1O] | 3-(2,4-DIHYDROXY-5-ETHYL- PHENYL)-4-(1-ISOPROPYL- INDOL-3-YL)-5-HYDROXY- [1,2,4] TRIAZOLE |
| 12 | 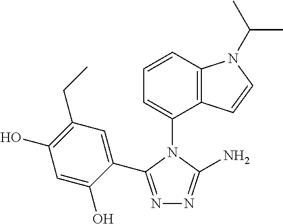 [SMILES: CCc1cc(-c2nnc(N)n2-c2cccc3c2ccn3C(C)C)c(O)cc1O] | 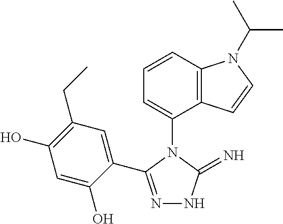 [SMILES: CCc1cc(-c2n[nH]c(=N)n2-c2cccc3c2ccn3C(C)C)c(O)cc1O] | 3-(2,4-DIHYDROXY-5-ETHYL- PHENYL)-4-(1-ISOPROPYL- INDOL-4-YL)-5-AMINO-[1,2,4] TRIAZOLE |
| 15 | 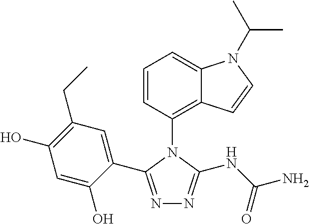 [SMILES: CCc1cc(-c2nnc(NC(N)=O)n2-c2cccc3c2ccn3C(C)C)c(O)cc1O] | | 3-(2,4-DIHYDROXY-5-ETHYL- PHENYL)-4-(1-ISOPROPYL- INDOL-4-YL)-5-UREIDO-[1,2,4] TRIAZOLE |
| 16 | 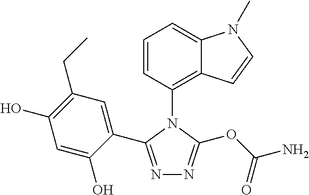 [SMILES: CCc1cc(-c2nnc(OC(N)=O)n2-c2cccc3c2ccn3C)c(O)cc1O] | | 3-(2,4-DIHYDROXY-5-ETHYL- PHENYL)-4-(1-METHYL-INDOL- 4-YL)-5-CARBAMOYLOXY- [1,2,4] TRIAZOLE |
| 17 | 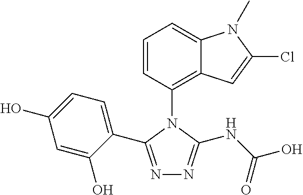 [SMILES: Cn1c(Cl)cc2c(-n3c(NC(=O)O)nnc3-c3ccc(O)cc3O)cccc21] | | 3-(2,4-DIHYDROXY-PHENYL)-4- (1-METHYL-2-CHLORO-INDOL- 4-YL)-5-CARBAMOYLOXY- [1,2,4] TRIAZOLE |
| 18 | 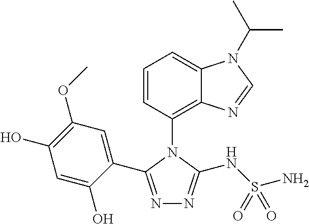 [SMILES: COc1cc(-c2nnc(NS(N)(=O)=O)n2-c2cccc3c2ncn3C(C)C)c(O)cc1O] | | 3-(2,4-DIHYDROXY-5- METHOXY-PHENYL)-4-(1- ISOPROPYL-BENZOIMIDAZOL- 4-YL)-5-(SULFAMOYLAMINO)- [1,2,4] TRIAZOLE |
| 20 | 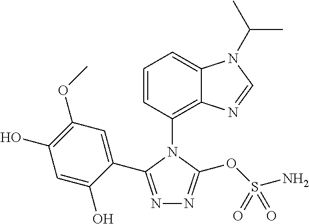 [SMILES: COc1cc(-c2nnc(OS(N)(=O)=O)n2-c2cccc3c2ncn3C(C)C)c(O)cc1O] | | 3-(2,4-DIHYDROXY-5- METHOXY-PHENYL)-4-(1- ISOPROPYL-BENZOIMIDAZOL- 4-YL)-5-(SULFAMOYLOXY)- [1,2,4] TRIAZOLE |
| 21 | 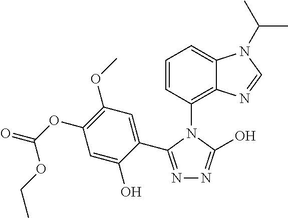 [SMILES: CCOC(=O)Oc1cc(O)c(-c2nnc(O)n2-c2cccc3c2ncn3C(C)C)cc1OC] | 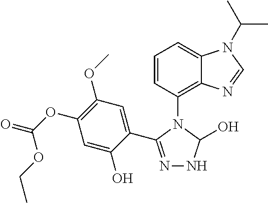 [SMILES: CCOC(=O)Oc1cc(O)c(C2=NNC(O)N2c2cccc3c2ncn3C(C)C)cc1OC] | 3-(2-HYDROXY-4- ETHOXYCARBONYOXY-5- METHOXY-PHENYL)-4-(1- ISOPROPYL-BENZOIMIDAZOL- 4-YL)-5-HYDROXY-[1,2,4] TRIAZOLE |
| 22 | 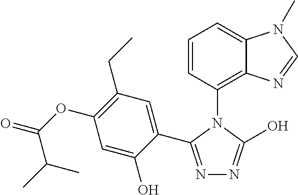 [SMILES: CCc1cc(-c2nnc(O)n2-c2cccc3c2ncn3C)c(O)cc1OC(=O)C(C)C] | 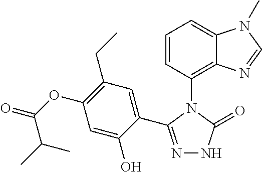 [SMILES: CCc1cc(-c2n[nH]c(=O)n2-c2cccc3c2ncn3C)c(O)cc1OC(=O)C(C)C] | 3-[2-HYDROXY-4- ISOBUTYRYLOXY-5-ETHYL- PHENYL]-4-(1-METHYL-BENZO- IMIDAZOL-4-YL)-5-HYDROXY- [1,2,4] TRIAZOLE |
| 23 | 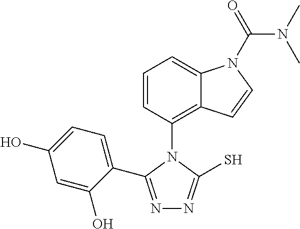 [SMILES: CN(C)C(=O)n1ccc2c(-n3c(S)nnc3-c3ccc(O)cc3O)cccc21] | 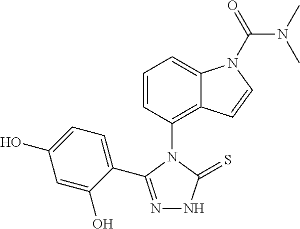 [SMILES: CN(C)C(=O)n1ccc2c(-n3c(-c4ccc(O)cc4O)n[nH]c3=S)cccc21] | 3-(2,4-DIHYDROXY-PHENYL)-4- (1-DIMETHYLCARBAMOYL- INDOL-4-YL)-5-MERCAPTO- [1,2,4] TRIAZOLE |
| 24 | 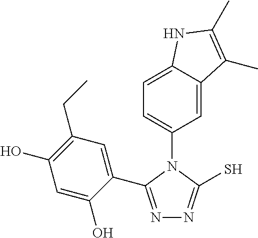 [SMILES: CCc1cc(-c2nnc(S)n2-c2ccc3[nH]c(C)c(C)c3c2)c(O)cc1O] | 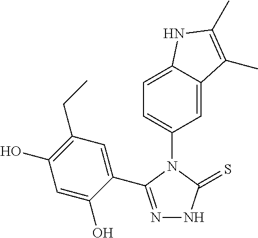 [SMILES: CCc1cc(-c2n[nH]c(=S)n2-c2ccc3[nH]c(C)c(C)c3c2)c(O)cc1O] | 3-(2,4-DIHYDROXY-5-ETHYL- PHENYL)-4-(2,3-DIMETHYL- INDOL-5-YL)-5-MERCAPTO- [1,2,4] TRIAZOLE |
| 25 | 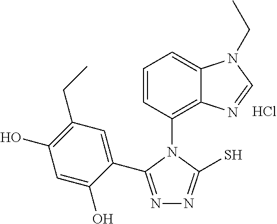 [SMILES: CCc1cc(-c2nnc(S)n2-c2cccc3c2ncn3CC)c(O)cc1O ; Cl] | 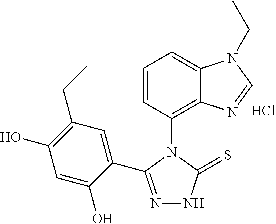 [SMILES: CCc1cc(-c2n[nH]c(=S)n2-c2cccc3c2ncn3CC)c(O)cc1O ; Cl] | 3-(2,4-DIHYDROXY-5-ETHYL- PHENYL)-4-(1-ETHYL-1H- BENZOIMIDAZOL-4-YL)-5- MERCAPTO-[1,2,4] TRIAZOLE, HCL SALT |
| 26 | 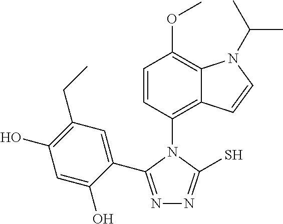 [SMILES: CCc1cc(-c2nnc(S)n2-c2ccc(OC)c3c2ccn3C(C)C)c(O)cc1O] | 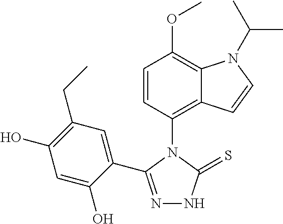 [SMILES: CCc1cc(-c2n[nH]c(=S)n2-c2ccc(OC)c3c2ccn3C(C)C)c(O)cc1O] | 3-(2,4-DIHYDROXY-5-ETHYL- PHENYL)-4-(1-ISOPROPYL-7- METHOXY-INDOL-4-YL)-5- MERCAPTO-[1,2,4] TRIAZOLE |
| 27 | 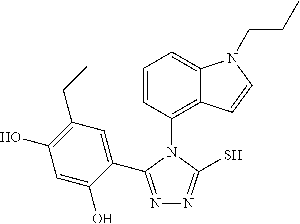 [SMILES: CCCn1ccc2c(-n3c(S)nnc3-c3cc(CC)c(O)cc3O)cccc21] | 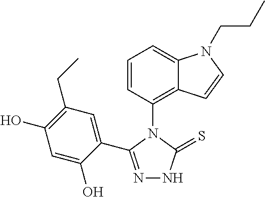 [SMILES: CCCn1ccc2c(-n3c(-c4cc(CC)c(O)cc4O)n[nH]c3=S)cccc21] | 3-(2,4-DIHYDROXY-5-ETHYL- PHENYL)-4-(1-PROPYL-INDOL- 4-YL)-5-MERCAPTO-[1,2,4] TRIAZOLE |
| 28 | 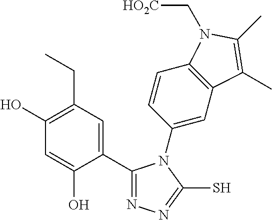 [SMILES: CCc1cc(-c2nnc(S)n2-c2ccc3c(c2)c(C)c(C)n3CC(=O)O)c(O)cc1O] | 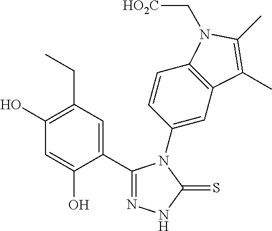 [SMILES: CCc1cc(-c2n[nH]c(=S)n2-c2ccc3c(c2)c(C)c(C)n3CC(=O)O)c(O)cc1O] | 3-(2,4-DIHYDROXY-5-ETHYL- PHENYL)-4-(1-ACETYL-2,3- DIMETHYL-INDOL-5-YL)-5- MERCAPTO-[1,2,4] TRIAZOLE |
| 29 | 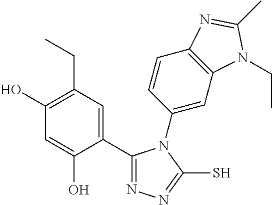 [SMILES: CCc1cc(-c2nnc(S)n2-c2ccc3nc(C)n(CC)c3c2)c(O)cc1O] | 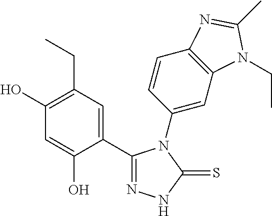 [SMILES: CCc1cc(-c2n[nH]c(=S)n2-c2ccc3nc(C)n(CC)c3c2)c(O)cc1O] | 3-(2,4-DIHYDROXY-5-ETHYL- PHENYL)-4-(2-METHYL-3- ETHYL-BENZIMIDAZOL-5-YL)- 5-MERCAPTO-[1,2,4] TRIAZOLE |
| 30 | 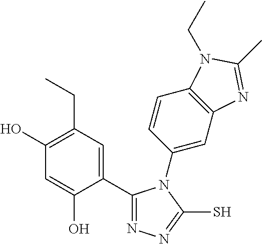 [SMILES: CCc1cc(-c2nnc(S)n2-c2ccc3c(c2)nc(C)n3CC)c(O)cc1O] | 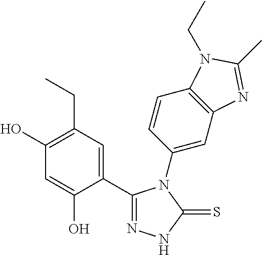 [SMILES: CCc1cc(-c2n[nH]c(=S)n2-c2ccc3c(c2)nc(C)n3CC)c(O)cc1O] | 3-(2,4-DIHYDROXY-5-ETHYL- PHENYL)-4-(1-ETHYL-2- METHYL-BENZIMIDAZOL-5- YL)-5-MERCAPTO-[1,2,4] TRIAZOLE |
| 31 | 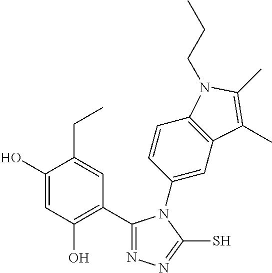 [SMILES: CCCn1c(C)c(C)c2cc(-n3c(S)nnc3-c3cc(CC)c(O)cc3O)ccc21] | 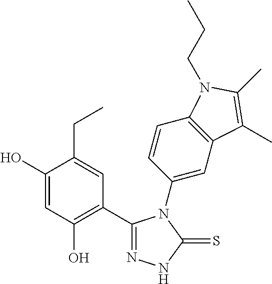 [SMILES: CCCn1c(C)c(C)c2cc(-n3c(-c4cc(CC)c(O)cc4O)n[nH]c3=S)ccc21] | 3-(2,4-DIHYDROXY-5-ETHYL- PHENYL)-4-(1-PROPYL-2,3- DIMETHYL-INDOL-5-YL)-5- MERCAPTO-[1,2,4] TRIAZOLE |
| 34 | 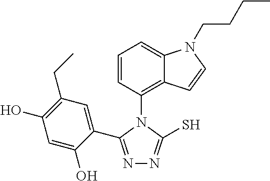 [SMILES: CCCCn1ccc2c(-n3c(S)nnc3-c3cc(CC)c(O)cc3O)cccc21] | 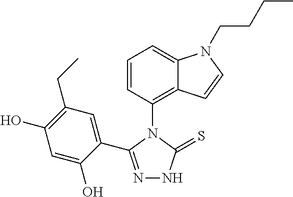 [SMILES: CCCCn1ccc2c(-n3c(-c4cc(CC)c(O)cc4O)n[nH]c3=S)cccc21] | 3-(2,4-DIHYDROXY-5-ETHYL- PHENYL)-4-(1-N-BUTYL-INDOL- 4-YL)-5-MERCAPTO-[1,2,4] TRIAZOLE |
| 35 | 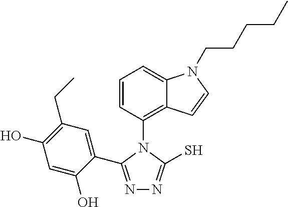 [SMILES: CCCCCn1ccc2c(-n3c(S)nnc3-c3cc(CC)c(O)cc3O)cccc21] | 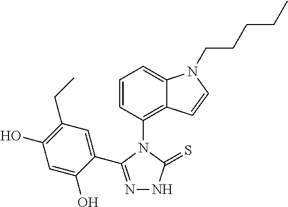 [SMILES: CCCCCn1ccc2c(-n3c(-c4cc(CC)c(O)cc4O)n[nH]c3=S)cccc21] | 3-(2,4-DIHYDROXY-5-ETHYL- PHENYL)-4-(1-N-PENTYL- INDOL-4-YL)-5-MERCAPTO- [1,2,4] TRIAZOLE |
| 36 | 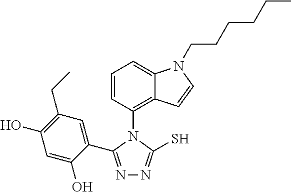 [SMILES: CCCCCCn1ccc2c(-n3c(S)nnc3-c3cc(CC)c(O)cc3O)cccc21] | 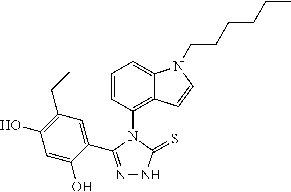 [SMILES: CCCCCCn1ccc2c(-n3c(-c4cc(CC)c(O)cc4O)n[nH]c3=S)cccc21] | 3-(2,4-DIHYDROXY-5-ETHYL- PHENYL)-4-(1-N-HEXYL-INDOL- 4-YL)-5-MERCAPTO-[1,2,4] TRIAZOLE |
| 37 | 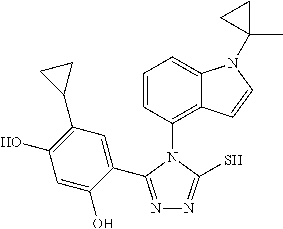 [SMILES: CC1(n2ccc3c(-n4c(S)nnc4-c4cc(C5CC5)c(O)cc4O)cccc32)CC1] | 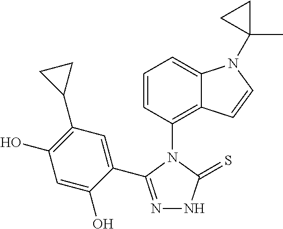 [SMILES: CC1(n2ccc3c(-n4c(-c5cc(C6CC6)c(O)cc5O)n[nH]c4=S)cccc32)CC1] | 3-(2,4-DIHYDROXY-5- CYCLOPROPYL-PHENYL)-4-(1- (1-METHYLCYCLOPROPYL)- INDOL-4-YL)-5-MERCAPTO- [1,2,4] TRIAZOLE |
| 38 | 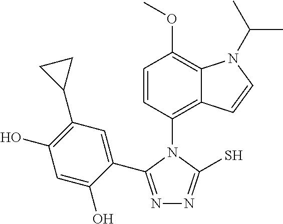 [SMILES: COc1ccc(-n2c(S)nnc2-c2cc(C3CC3)c(O)cc2O)c2ccn(C(C)C)c12] | 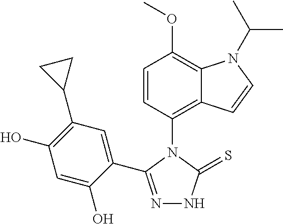 [SMILES: COc1ccc(-n2c(-c3cc(C4CC4)c(O)cc3O)n[nH]c2=S)c2ccn(C(C)C)c12] | 3-(2,4-DIHYDROXY-5- CYCLOPROPYL-PHENYL)-4-(1- ISOPROPYL-7-METHOXY- INDOL-4-YL)-5-MERCAPTO- [1,2,4] TRIAZOLE |
| 39 | 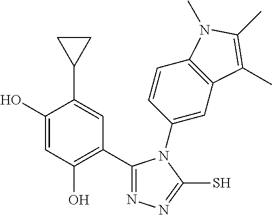 [SMILES: Cc1c(C)n(C)c2ccc(-n3c(S)nnc3-c3cc(C4CC4)c(O)cc3O)cc12] | 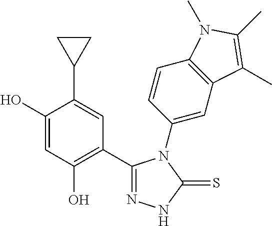 [SMILES: Cc1c(C)n(C)c2ccc(-n3c(-c4cc(C5CC5)c(O)cc4O)n[nH]c3=S)cc12] | 3-(2,4-DIHYDROXY-5- CYCLOPROPYL-PHENYL)-4- (1,2,3-TRIMETHYL-INDOL-5 YL)-5-MERCAPTO-[1,2,4] TRIAZOLE |
| 40 | 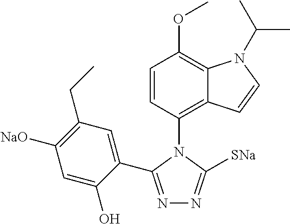 | | 3-(2,4-DIHYDROXY-5-ETHYL- PHENYL)-4-(1-ISOPROPYL-7- METHOXY-INDOL-4-YL)-5- MERCAPTO-[1,2,4] TRIAZOLE DISODIUM SALT |
| 41 | 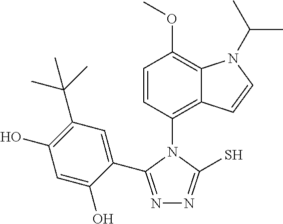 [SMILES: COc1ccc(-n2c(S)nnc2-c2cc(C(C)(C)C)c(O)cc2O)c2ccn(C(C)C)c12] | 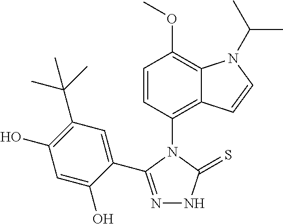 [SMILES: COc1ccc(-n2c(-c3cc(C(C)(C)C)c(O)cc3O)n[nH]c2=S)c2ccn(C(C)C)c12] | 3-(2,4-DIHYDROXY-5-TERT- BUTYL-PHENYL)-4-(1- ISOPROPYL-7-METHOXY- INDOL-4-YL)-5-MERCAPTO- [1,2,4] TRIAZOLE |
| 42 | 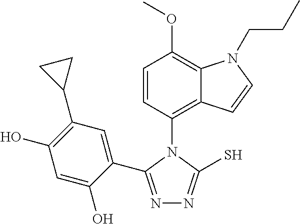 [SMILES: CCCn1ccc2c(-n3c(S)nnc3-c3cc(C4CC4)c(O)cc3O)ccc(OC)c21] | 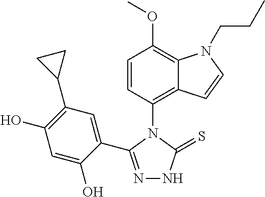 [SMILES: CCCn1ccc2c(-n3c(-c4cc(C5CC5)c(O)cc4O)n[nH]c3=S)ccc(OC)c21] | 3-(2,4-DIHYDROXY-5- CYCLOPROPYL-PHENYL)-4-(1- PROPYL-7-METHOXY-INDOL-4- YL)-5-MERCAPTO-[1,2,4] TRIAZOLE |
| 43 | 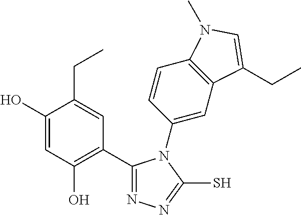 [SMILES: CCc1cc(-c2nnc(S)n2-c2ccc3c(c2)c(CC)cn3C)c(O)cc1O] | 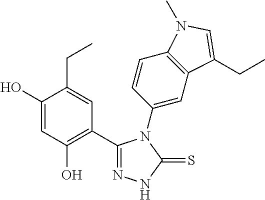 [SMILES: CCc1cc(-c2n[nH]c(=S)n2-c2ccc3c(c2)c(CC)cn3C)c(O)cc1O] | 3-(2,4-DIHYDROXY-5-ETHYL- PHENYL)-4-(1-METHYL-3- ETHYL-INDOL-5-YL)-5- MERCAPTO-[1,2,4] TRIAZOLE |
| 44 | 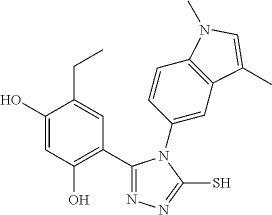 [SMILES: CCc1cc(-c2nnc(S)n2-c2ccc3c(c2)c(C)cn3C)c(O)cc1O] | 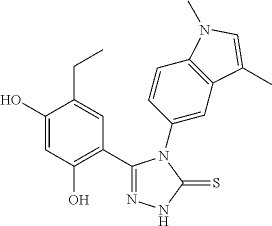 [SMILES: CCc1cc(-c2n[nH]c(=S)n2-c2ccc3c(c2)c(C)cn3C)c(O)cc1O] | 3-(2,4-DIHYDROXY-5-ETHYL- PHENYL)-4-(1,3-DIMETHYL- INDOL-5-YL)-5-MERCAPTO- [1,2,4] TRIAZOLE |
| 45 | 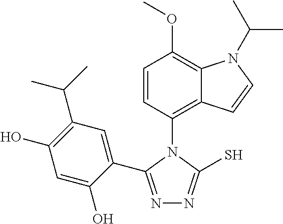 [SMILES: COc1ccc(-n2c(S)nnc2-c2cc(C(C)C)c(O)cc2O)c2ccn(C(C)C)c12] | 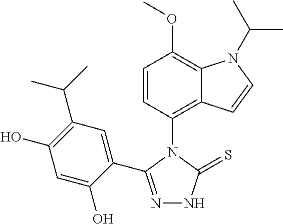 [SMILES: COc1ccc(-n2c(-c3cc(C(C)C)c(O)cc3O)n[nH]c2=S)c2ccn(C(C)C)c12] | 3-(2,4-DIHYDROXY-5- ISOPROPYL-PHENYL)-4-(1- ISOPROPYL-7-METHOXY- INDOL-4-YL)-5-MERCAPTO- [1,2,4] TRIAZOLE |
| 46 | 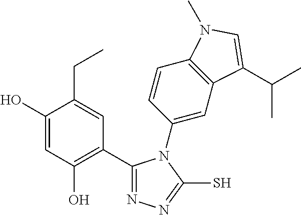 [SMILES: CCc1cc(-c2nnc(S)n2-c2ccc3c(c2)c(C(C)C)cn3C)c(O)cc1O] | 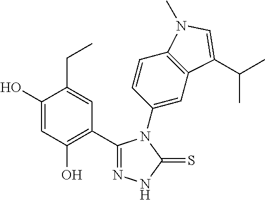 [SMILES: CCc1cc(-c2n[nH]c(=S)n2-c2ccc3c(c2)c(C(C)C)cn3C)c(O)cc1O] | 3-(2,4-DIHYDROXY-5-ETHYL- PHENYL)-4-(1-METHYL-3- ISOPROPYL-INDOL-5-YL)-5- MERCAPTO-[1,2,4] TRIAZOLE |
| 48 | 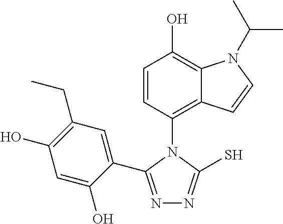 [SMILES: CCc1cc(-c2nnc(S)n2-c2ccc(O)c3c2ccn3C(C)C)c(O)cc1O] | 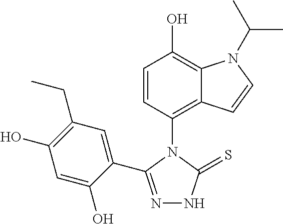 [SMILES: CCc1cc(-c2n[nH]c(=S)n2-c2ccc(O)c3c2ccn3C(C)C)c(O)cc1O] | 3-(2,4-DIHYDROXY-5-ETHYL- PHENYL)-4-(1-ISOPROPYL-7- HYDROXY-INDOL-4-YL)-5- MERCAPTO-[1,2,4] TRIAZOLE |
| 49 | 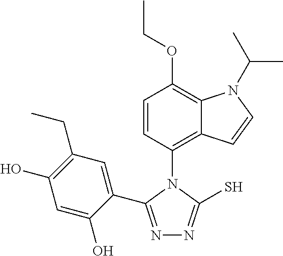 [SMILES: CCOc1ccc(-n2c(S)nnc2-c2cc(CC)c(O)cc2O)c2ccn(C(C)C)c12] | 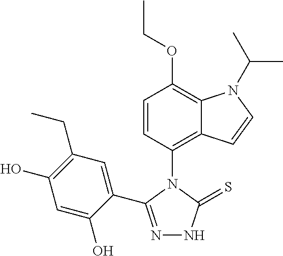 [SMILES: CCOc1ccc(-n2c(-c3cc(CC)c(O)cc3O)n[nH]c2=S)c2ccn(C(C)C)c12] | 3-(2,4-DIHYDROXY-5-ETHYL- PHENYL)-4-(1-ISOPROPYL-7- ETHOXY-INDOL-4-YL)-5- MERCAPTO-[1,2,4] TRIAZOLE |
| 50 | 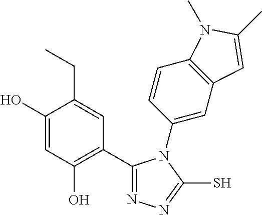 [SMILES: CCc1cc(-c2nnc(S)n2-c2ccc3c(c2)cc(C)n3C)c(O)cc1O] | 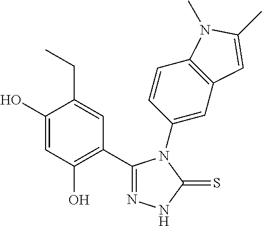 [SMILES: CCc1cc(-c2n[nH]c(=S)n2-c2ccc3c(c2)cc(C)n3C)c(O)cc1O] | 3-(2,4-DIHYDROXY-5-ETHYL- PHENYL)-4-(1,2-DIMETHYL- INDOL-5-YL)-5-MERCAPTO- [1,2,4] TRIAZOLE |
| 51 | 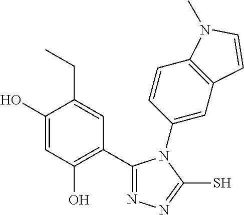 [SMILES: CCc1cc(-c2nnc(S)n2-c2ccc3c(ccn3C)c2)c(O)cc1O] | 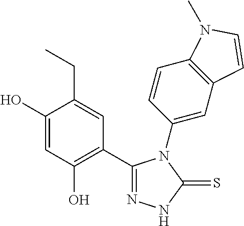 [SMILES: CCc1cc(-c2n[nH]c(=S)n2-c2ccc3c(ccn3C)c2)c(O)cc1O] | 3-(2,4-DIHYDROXY-5-ETHYL- PHENYL)-4-(N-METHYL- INDOL-5-YL)-5-MERCAPTO- [1,2,4] TRIAZOLE |
| 55 | 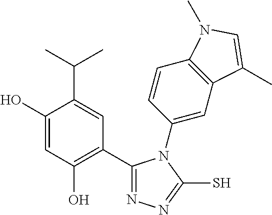 [SMILES: Cc1cn(C)c2ccc(-n3c(S)nnc3-c3cc(C(C)C)c(O)cc3O)cc12] | 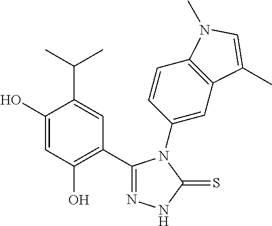 [SMILES: Cc1cn(C)c2ccc(-n3c(-c4cc(C(C)C)c(O)cc4O)n[nH]c3=S)cc12] | 3-(2,4-DIHYDROXY-5- ISOPROPYL-PHENYL)-4-(1,3- DIMETHYL-INDOL-5-YL)-5- MERCAPTO-[1,2,4] TRIAZOLE |
| 56 | 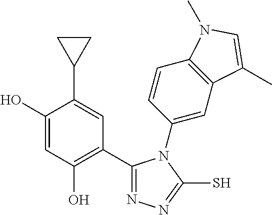 [SMILES: Cc1cn(C)c2ccc(-n3c(S)nnc3-c3cc(C4CC4)c(O)cc3O)cc12] | 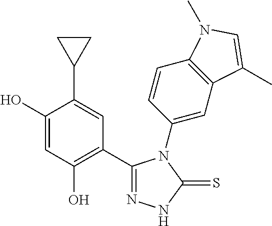 [SMILES: Cc1cn(C)c2ccc(-n3c(-c4cc(C5CC5)c(O)cc4O)n[nH]c3=S)cc12] | 3-(2,4-DIHYDROXY-5- CYCLOPROPYL-PHENYL)-4-(1,3- DIMETHYL-INDOL-5-YL)-5- MERCAPTO-[1,2,4] TRIAZOLE |
| 57 | 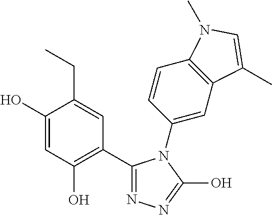 [SMILES: CCc1cc(-c2nnc(O)n2-c2ccc3c(c2)c(C)cn3C)c(O)cc1O] | 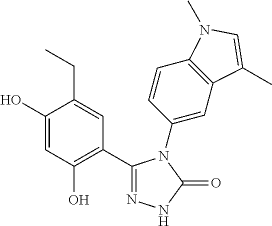 [SMILES: CCc1cc(-c2n[nH]c(=O)n2-c2ccc3c(c2)c(C)cn3C)c(O)cc1O] | 3-(2,4-DIHYDROXY-5-ETHYL- PHENYL)-4-(1,3-DIMETHYL- INDOL-5-YL)-5-HYDROXY- [1,2,4] TRIAZOLE |
| 58 | 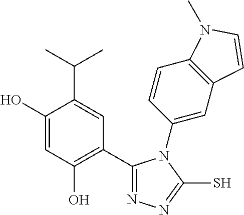 [SMILES: CC(C)c1cc(-c2nnc(S)n2-c2ccc3c(ccn3C)c2)c(O)cc1O] | 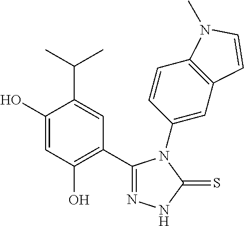 [SMILES: CC(C)c1cc(-c2n[nH]c(=S)n2-c2ccc3c(ccn3C)c2)c(O)cc1O] | 3-(2,4-DIHYDROXY-5- ISOPROPYL-PHENYL)-4-(N- METHYL-INDOL-5-YL)-5- MERCAPTO-[1,2,4] TRIAZOLE |
| 59 | 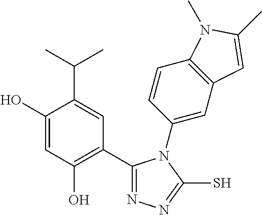 [SMILES: Cc1cc2cc(-n3c(S)nnc3-c3cc(C(C)C)c(O)cc3O)ccc2n1C] | 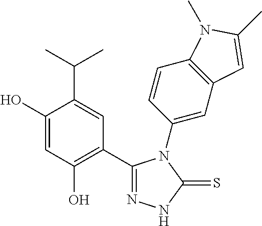 [SMILES: Cc1cc2cc(-n3c(-c4cc(C(C)C)c(O)cc4O)n[nH]c3=S)ccc2n1C] | 3-(2,4-DIHYDROXY-5- ISOPROPYL-PHENYL)-4-(1,2- DIMETHYL-INDOL-5-YL)-5- MERCAPTO-[1,2,4] TRIAZOLE |
| 60 | 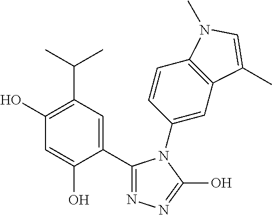 [SMILES: Cc1cn(C)c2ccc(-n3c(O)nnc3-c3cc(C(C)C)c(O)cc3O)cc12] | 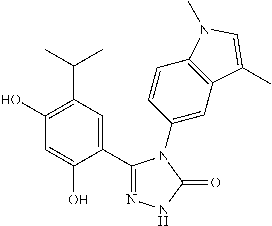 [SMILES: Cc1cn(C)c2ccc(-n3c(-c4cc(C(C)C)c(O)cc4O)n[nH]c3=O)cc12] | 3-(2,4-DIHYDROXY-5- ISOPROPYL-PHENYL)-4-(1,3- DIMETHYL-INDOL-5-YL)-5- HYDROXY-[1,2,4] TRIAZOLE |
| 62 | 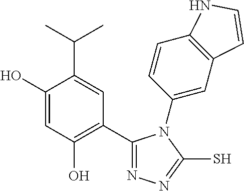 [SMILES: CC(C)c1cc(-c2nnc(S)n2-c2ccc3[nH]ccc3c2)c(O)cc1O] | 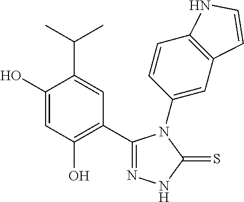 [SMILES: CC(C)c1cc(-c2n[nH]c(=S)n2-c2ccc3[nH]ccc3c2)c(O)cc1O] | 3-(2,4-DIHYDROXY-5- ISOPROPYL-PHENYL)-4-(1H- INDOL-5-YL)-5-MERCAPTO- [1,2,4] TRIAZOLE |
| 63 | 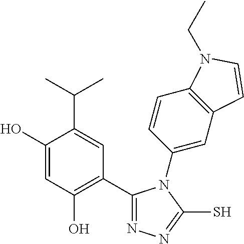 [SMILES: CCn1ccc2cc(-n3c(S)nnc3-c3cc(C(C)C)c(O)cc3O)ccc21] | 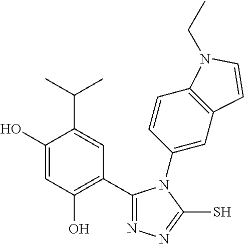 [SMILES: CCn1ccc2cc(-n3c(S)nnc3-c3cc(C(C)C)c(O)cc3O)ccc21] | 3-(2,4-DIHYDROXY-5- ISOPROPYL-PHENYL)-4-(1- ETHYL-INDOL-5-YL)-5- MERCAPTO-[1,2,4] TRIAZOLE |
| 64 | 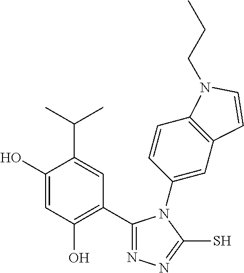 [SMILES: CCCn1ccc2cc(-n3c(S)nnc3-c3cc(C(C)C)c(O)cc3O)ccc21] | 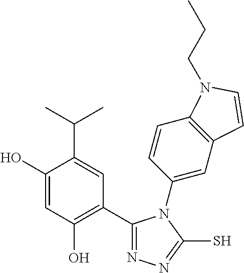 [SMILES: CCCn1ccc2cc(-n3c(S)nnc3-c3cc(C(C)C)c(O)cc3O)ccc21] | 3-(2,4-DIHYDROXY-5- ISOPROPYL-PHENYL)-4-(1- PROPYL-INDOL-5-YL)-5- MERCAPTO-[1,2,4] TRIAZOLE |
| 65 | 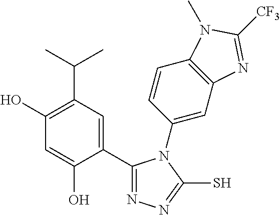 [SMILES: CC(C)c1cc(-c2nnc(S)n2-c2ccc3c(c2)nc(C(F)(F)F)n3C)c(O)cc1O] | 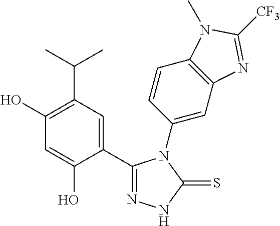 [SMILES: CC(C)c1cc(-c2n[nH]c(=S)n2-c2ccc3c(c2)nc(C(F)(F)F)n3C)c(O)cc1O] | 3-(2,4-DIHYDROXY-5- ISOPROPYL-PHENYL)-4-(1- METHYL-2- TRIFLUOROMETHYL- BENZIMIDAZOL-5-YL)-5- MERCAPTO-[1,2,4] TRIAZOLE |
| 66 | 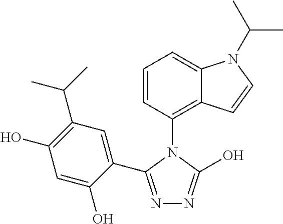 [SMILES: CC(C)c1cc(-c2nnc(O)n2-c2cccc3c2ccn3C(C)C)c(O)cc1O] | 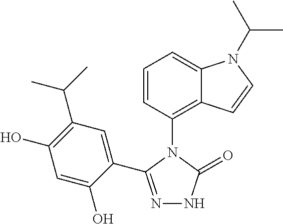 [SMILES: CC(C)c1cc(-c2n[nH]c(=O)n2-c2cccc3c2ccn3C(C)C)c(O)cc1O] | 3-(2,4-DIHYDROXY-5- ISOPROPYL-PHENYL)-4-(1- ISOPROPYL-INDOL-4-YL)-5- HYDROXY-[1,2,4] TRIAZOLE |
| 67 | 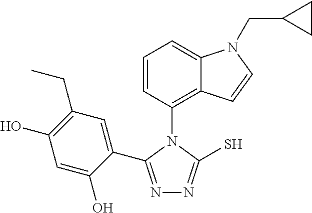 [SMILES: CCc1cc(-c2nnc(S)n2-c2cccc3c2ccn3CC2CC2)c(O)cc1O] | 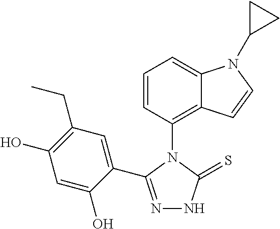 [SMILES: CCc1cc(-c2n[nH]c(=S)n2-c2cccc3c2ccn3C2CC2)c(O)cc1O] | 3-(2,4-DIHYDROXY-5-ETHYL- PHENYL)-4-(1- (CYCLOPROPYLMETHYL)- INDOL-4-YL)-5-MERCAPTO- [1,2,4] TRIAZOLE |

TABLE 2
Compounds according to Formula (Ia)

| NO. | STRUCTURE | TAUTOMERIC STRUCTURE | NAME |
|---|---|---|---|
| 1A | 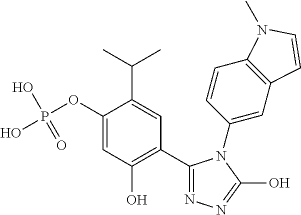 [SMILES: CC(C)c1cc(-c2nnc(O)n2-c2ccc3c(ccn3C)c2)c(O)cc1OP(=O)(O)O] | 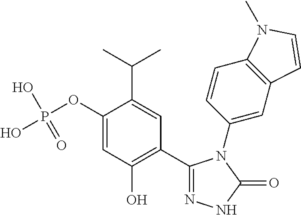 [SMILES: CC(C)c1cc(-c2n[nH]c(=O)n2-c2ccc3c(ccn3C)c2)c(O)cc1OP(=O)(O)O] | 5-HYDROXY-4-(5- HYDROXY-4-(1- METHYL-1H-INDOL-5- YL)-4H-1,2,4-TRIAZOL- 3-YL)-2- ISOPROPYLPHENYL DIHYDROGEN PHOSPHATE |
| 2A | 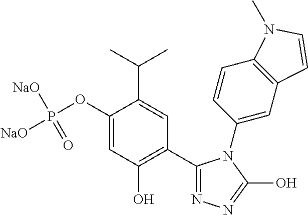 | 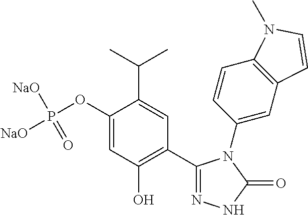 | SODIUM 5-HYDROXY-4- (5-HYDROXY-4-(1- METHYL-1H-INDOL-5- YL)-4H-1,2,4-TRIAZOL- 3-YL)-2- ISOPROPYLPHENYL PHOSPHATE |
| 3A | 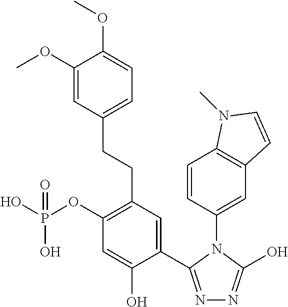 [SMILES: COc1ccc(CCc2cc(-c3nnc(O)n3-c3ccc4c(ccn4C)c3)c(O)cc2OP(=O)(O)O)cc1OC] | 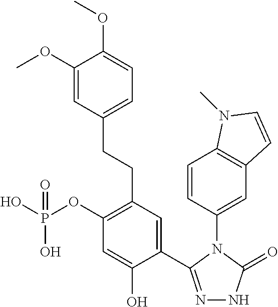 [SMILES: COc1ccc(CCc2cc(-c3n[nH]c(=O)n3-c3ccc4c(ccn4C)c3)c(O)cc2OP(=O)(O)O)cc1OC] | 2-(3,4- DIMETHOXYPHENETHYL)- 5-HYDROXY-4-(5- HYDROXY-4-(1- METHYL-1H-INDOL-5- YL)-4H-1,2,4-TRIAZOL- 3-YL)PHENYL DIHYDROGEN PHOSPHATE |
| 4A | 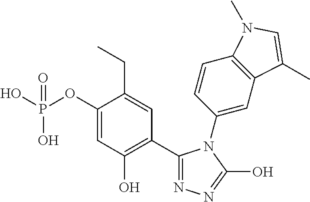 [SMILES: CCc1cc(-c2nnc(O)n2-c2ccc3c(c2)c(C)cn3C)c(O)cc1OP(=O)(O)O] | 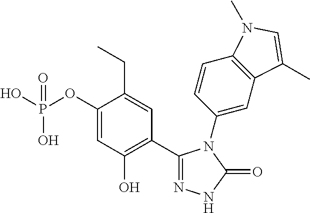 [SMILES: CCc1cc(-c2n[nH]c(=O)n2-c2ccc3c(c2)c(C)cn3C)c(O)cc1OP(=O)(O)O] | 4-(4-(1,3-DIMETHYL- 1H-INDOL-5-YL)-5- HYDROXY-4H-1,2,4- TRIAZOL-3-YL)-2- ETHYL-5- HYDROXYPHENYL DIHYDROGEN PHOSPHATE |

The Hsp90 inhibitory compounds used in the disclosed methods can be prepared according to the procedures disclosed in U.S. Patent Publication No. 2006/0,167,070, and WO2009/023,211.

These triazolone compounds typically can form a tautomeric structure as shown below and as exemplified by the tautomeric structures shown in Tables 1 and 2:

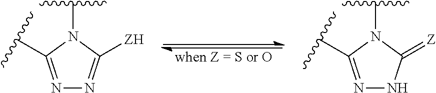

Other Hsp90 inhibitors include geldanamycin derivatives, e.g., a benzoquinone or hydroquinone ansamycin HSP90 inhibitor such as IPI-493 (CAS No. 64202-81-9) and/or IPI-504 (CAS No. 857402-63-2); 17-AAG CAS No. 75747-14-7), BIIB-021 (CNF-2024, CAS No. 848695-25-0), BIIB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), or PF-04928473 (SNX-2112, CAS No. 945626-71-1).

The method described herein includes treating a subject with cancer with a mutation in ROS protein, comprising the steps of identifying a subject with cancer with a mutation in ROS, and administering to the subject an effective amount of an Hsp90 inhibitor. In one embodiment, the cancer is non-small cell lung cancer. In one embodiment, the cancer is glioblastoma. In one embodiment, the cancer is brain cancer. In one embodiment, the cancer is head and neck cancer. In one embodiment, the cancer is stomach cancer. In one embodiment, the cancer is breast cancer. In one embodiment, the cancer is liver cancer. In one embodiment, the cancer is colon cancer. In one embodiment, the cancer is lung cancer. In one embodiment, the cancer is kidney cancer.

In an embodiment, the method includes treating a subject with cancer with a mutation in ROS, comprising the steps of identifying a subject with cancer with a mutation in ROS, and administering to the subject an effective amount of an Hsp90 inhibitor according to formulae (I) or (Ia) or a compound in Tables 1 or 2. In one embodiment, the Hsp90 inhibitor is ganetespib. In one embodiment, the Hsp90 inhibitor is compound 1A.

In one embodiment, the method includes treating a subject with non-small cell lung cancer with a mutation in ROS comprising identifying a subject with NSCLC with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with non-small cell lung cancer with a mutation in ROS comprising identifying a subject with NSCLC with a mutation in ROS and administering an effective amount of an Hsp90 inhibitor according to formulae (I) or (Ia). In one embodiment, the Hsp90 inhibitor is compound 1A and the effective amount is from about 100 mg/m² to about 500 mg/m².

In one embodiment, the method includes treating a subject with non-small cell lung cancer with a mutation in ROS comprising identifying a subject with NSCLC harboring a v-ROS fusion protein and administering an effective amount of an Hsp90 inhibitor according to formulae (I) or (Ia). In one embodiment, the Hsp90 inhibitor is ganetespib and the effective amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with non-small cell lung cancer with a mutation in ROS comprising identifying a subject with NSCLC harboring a v-ROS fusion protein and administering an effective amount of an Hsp90 inhibitor according to formulae (I) or (Ia). In one embodiment, the Hsp90 inhibitor is compound 1A and the effective amount is from about 100 m g/m² to about 500 mg/m².

In one embodiment, the method includes treating a subject with non-small cell lung cancer with a mutation in ROS comprising identifying a subject with NSCLC harboring an Mcf3 fusion protein and administering an effective amount of an Hsp90 inhibitor according to formulae (I) or (Ia). In one embodiment, the Hsp90 inhibitor is ganetespib and the effective amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with non-small cell lung cancer with a mutation in ROS comprising identifying a subject with NSCLC harboring an Mcf3 fusion protein and administering an effective amount of an Hsp90 inhibitor according to formulae (I) or (Ia). In one embodiment, the Hsp90 inhibitor is compound 1A and the effective amount is from about 100 mg/m² to about 500 mg/m².

In one embodiment, the method includes treating a subject with non-small cell lung cancer with a mutation in ROS comprising identifying a subject with NSCLC harboring a FIG-ROS fusion protein and administering an effective amount of an Hsp90 inhibitor according to formulae (I) or (Ia). In one embodiment, the Hsp90 inhibitor is ganetespib and the effective amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes a subject with non-small cell lung cancer with a mutation in ROS comprising identifying a subject with NSCLC harboring a FIG-ROS fusion protein and administering an effective amount of an Hsp90 inhibitor according to formulae (I) or (Ia). In one embodiment, the Hsp90 inhibitor is compound 1A and the effective amount is from about 100 mg/m² to about 500 mg/m².

In one embodiment, the method includes treating a subject with non-small cell lung cancer with a mutation in ROS comprising identifying a subject with NSCLC harboring an SLC34A2-ROS fusion protein and administering an effective amount of an Hsp90 inhibitor according to formulae (I) or (Ia). In one embodiment, the Hsp90 inhibitor is ganetespib and the effective amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with non-small cell lung cancer with a mutation in ROS comprising identifying a subject with NSCLC harboring an SLC34A2-ROS fusion protein and administering an effective amount of an Hsp90 inhibitor according to formulae (I) or (Ia). In one embodiment, the Hsp90 inhibitor is compound 1A and the effective amount is from about 100 mg/m² to about 500 mg/m².

In one embodiment, the method includes treating a subject with non-small cell lung cancer with a mutation in ROS comprising identifying a subject with NSCLC harboring a CD74-ROS fusion protein and administering an effective amount of an Hsp90 inhibitor according to formulae (I) or (Ia). In one embodiment, the Hsp90 inhibitor is ganetespib and the effective amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with non-small cell lung cancer with a mutation in ROS comprising identifying a subject with NSCLC harboring a CD74-ROS fusion protein and administering an effective amount of an Hsp90 inhibitor according to formulae (I) or (Ia). In one embodiment, the Hsp90 inhibitor is compound 1A and the effective amount is from about 100 mg/m² to about 500 mg/m².

In one embodiment, the method includes treating a subject with glioblastoma with a mutation in ROS comprising identifying a subject with glioblastoma with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with glioblastoma with a mutation in ROS comprising identifying a subject with glioblastoma with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with lung cancer with a mutation in ROS comprising identifying a subject with lung cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with lung cancer with a mutation in ROS comprising identifying a subject with lung cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with head and neck cancer with a mutation in ROS comprising identifying a subject with head and neck cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with head and neck cancer with a mutation in ROS comprising identifying a subject with head and neck cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with brain cancer with a mutation in ROS comprising identifying a subject with brain cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with brain cancer with a mutation in ROS comprising identifying a subject with brain cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with stomach cancer with a mutation in ROS comprising identifying a subject with stomach cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with stomach cancer with a mutation in ROS comprising identifying a subject with stomach cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with breast cancer with a mutation in ROS comprising identifying a subject with breast cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with breast cancer with a mutation in ROS comprising identifying a subject with breast cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with liver cancer with a mutation in ROS comprising identifying a subject with liver cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with liver cancer with a mutation in ROS comprising identifying a subject with liver cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with colon cancer with a mutation in ROS comprising identifying a subject with colon cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with colon cancer with a mutation in ROS comprising identifying a subject with colon cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In one embodiment, the method includes treating a subject with kidney cancer with a mutation in ROS comprising identifying a subject with kidney cancer with a mutation in ROS and administering an effective amount of an Hsp90 inhibitor according to formulae (I) or (Ia). In one embodiment, the Hsp90 inhibitor is ganetespib and the amount is from about 100 mg/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly.

In one embodiment, the method includes treating a subject with kidney cancer with a mutation in ROS comprising identifying a subject with kidney cancer with a mutation in ROS and administering an effective amount of an Hsp90 inhibitor according to formulae (I) or (Ia). In one embodiment, the Hsp90 inhibitor is compound 1A and the amount is from about 100 mg/m² to about 500 mg/m².

In any one of the above-mentioned embodiments, ganetespib or 1A may be administered in combination with one or more additional therapeutic agents. The one or more additional therapeutic agents may be BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline.

In one aspect, the method also includes treating cancer in a subject wherein the cancer has been previously treated with an anticancer agent and is no longer responsive to the earlier treatment (resistant to the treatment). In one embodiment, the previous anticancer agent may be crizotinib.

In one embodiment, the method includes treating NSCLC in a patient wherein the patient has been previously treated with one or more other anticancer agent and is not responsive to the earlier treatments (resistant to further treatment) by administering an amount of from about 100 mg/m² to about 500 mg/m² of ganetespib. In one embodiment, the previous anticancer agent may be crizotinib.

In one embodiment, the method includes treating NSCLC in a patient wherein the patient has been previously treated with one or more other anticancer agent and is no longer responsive to the earlier treatments (resistant to further treatment) by administering an amount of from about 100 mg/m² to about 500 mg/m² of compound 1A. In one embodiment, the previous anticancer agent may be crizotinib.

In an embodiment, the method of treating a subject with cancer with a mutation in ROS, includes a) identifying a subject with a mutation in ROS; and b) administering to the subject an effective amount of a compound of formulae (I) or (Ia), or a compound in Table 1 or 2, or a pharmaceutically acceptable salt or tautomer thereof. In an embodiment, the compound is ganetespib. In an embodiment, the compound is 1A. In an embodiment, the method further comprises administering one or more additional anticancer drugs. In an embodiment, the one or more drugs may be BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline.

In an embodiment, the method of treating a subject with NSCLC with a mutation in ROS, includes a) identifying a subject with a mutation in ROS; and b) administering to the subject an effective amount of a compound of formulae (I) or (Ia), or a compound in Table 1 or 2, or a pharmaceutically acceptable salt or tautomer thereof. In an embodiment, the compound is ganetespib. In an embodiment, the compound is 1A. In an embodiment, the method further comprises administering one or more additional anticancer drugs. In an embodiment, the one or more drugs may be BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline.

In an embodiment, the method of treating a subject with glioblastoma with a mutation in ROS, includes a) identifying a subject with a mutation in ROS; and b) administering to the subject an effective amount of a compound of formulae (I) or (Ia), or a compound in Table 1 or 2, or a pharmaceutically acceptable salt or tautomer thereof. In an embodiment, the compound is ganetespib. In an embodiment, the compound is 1A. In an embodiment, the method further comprises administering one or more additional anticancer drugs. In an embodiment, the one or more drugs may be BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline.

In one embodiment, the method also includes treating a subject with cancer with a RET mutation, or rearrangement comprising identifying a subject with cancer with a mutation in RET and administering an effective amount of an Hsp90 inhibitor according to formulae (I) or (Ia) or a compound listed in Table 1 or 2. In one embodiment, the Hsp90 inhibitor is ganetespib and the amount is from about 100 m g/m² to about 500 mg/m². In one embodiment, the Hsp90 inhibitor is ganetespib and the amount is about 200 mg/m². In one embodiment, the Hsp90 inhibitor ganetespib is administered at about 200 mg/m² once weekly. In one embodiment, the cancer is non-small cell lung cancer. In one embodiment, the cancer is thyroid cancer. In one embodiment, the cancer is lung adenocarcinoma. In an embodiment, the method further comprises administering one or more additional anticancer drugs. In an embodiment, the one or more drugs may be BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, or tetracycline.

In one embodiment, the method includes treating a subject with cancer with a mutation in RET comprising identifying a subject with cancer with a mutation in RET and administering an effective amount of an Hsp90 inhibitor according to formulae (I) or (Ia) or a compound listed in Table 1 or 2. In one embodiment, the Hsp90 inhibitor is compound 1A and the amount is from about 100 mg/m² to about 500 mg/m². In one embodiment, the cancer is non-small cell lung cancer. In one embodiment, the cancer is thyroid cancer. In one embodiment, the cancer is lung adenocarcinoma.

The identification of the presence of alteration, mutation, or rearrangement in a ROS gene or gene product in a sample from a subject can be achieved through known methods and procedures as disclosed in the literature. See, e.g., Ibrahim Mustafa El-Deeb et al, Medicinal Research Reviews, 31,No. 5, 794-818, 2011; Charest et al, Cancer Res 2006; 66:7473-7481; Fei Li et al, Cell Research (2012):1-4; Chenguang Li et al, PLoS One. 2011; 6(11):e28204; Ting-Lei Gu et al, PLoS One. 2011; 6(1):e15640; and the references cited in the above-identified references. Some of the specific examples for the detection of alteration, or mutation or rearrangement in a ROS gene or gene product are also shown in the Examples of this application.

The therapeutic agents described herein can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

The triazolone compounds described herein can be formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In specific embodiment, the amount of the compound of formulae (I) or (Ia) administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In one embodiment, the amount of the compound of formula (I) administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of the compound of formula (I) administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m². The compound of formulae (I) or (Ia) can be administered 1, 2, 3, 4 or more times daily, or once every 2, 3, 4, 5, 6 or 7 days, or once weekly, once every two weeks, once every three weeks or once monthly.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the method includes preventing, treating, managing, or ameliorating a proliferative disorders, such as cancer, or one or more symptoms thereof, comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

The invention also provides the use of a compound of structural formula (I) or (Ia) or a compound in Tables 1 or 2 or a pharmaceutically acceptable salt thereof for the manufacture of a medicament for the treatment of a subject with a cancer with a mutation in ROS. The invention further provides the use of a compound of structural formula (I) or (Ia) or a compound in Tables 1 or 2 or a pharmaceutically acceptable salt thereof for the manufacture of a medicament for the treatment of a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of a compound of structural formula (I) or (Ia) or a compound in Tables 1 or 2 or a pharmaceutically acceptable salt thereof for the manufacture of a medicament for the treatment of a subject with a NSCLC with a mutation in ROS. The invention further provides the use of a compound of structural formula (I) or (Ia) or a compound in Tables 1 or 2 or a pharmaceutically acceptable salt thereof for the manufacture of a medicament for the treatment of a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of a compound of structural formula (I) or (Ia) or a compound in Tables 1 or 2 or a pharmaceutically acceptable salt thereof for the manufacture of a medicament for the treatment of a subject with a glioblastoma with a mutation in ROS. The invention further provides the use of a compound of structural formula (I) or (Ia) or a compound in Tables 1 or 2 or a pharmaceutically acceptable salt thereof for the manufacture of a medicament for the treatment of a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of a compound of structural formula (I) or (Ia) or a compound in Tables 1 or 2 or a pharmaceutically acceptable salt thereof for the manufacture of a medicament for the treatment of a subject with a head and neck cancer, colon cancer, breast cancer, liver cancer, stomach cancer, or kidney cancer with a mutation in ROS. The invention further provides the use of a compound of structural formula (I) or (Ia) or a compound in Tables 1 or 2 or a pharmaceutically acceptable salt thereof for the manufacture of a medicament for the treatment of a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a cancer with a mutation in ROS. The invention further provides the use of ganetespib or a pharmaceutically acceptable salt thereof for the manufacture of a medicament for the treatment of a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a NSCLC with a mutation in ROS. The invention further provides the use of ganetespib or a pharmaceutically acceptable salt thereof for the manufacture of a medicament for the treatment of a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a glioblastoma with a mutation in ROS. The invention further provides the use of ganetespib or a pharmaceutically acceptable salt thereof for the manufacture of a medicament for the treatment of a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a head and neck cancer, colon cancer, breast cancer, liver cancer, stomach cancer, or kidney cancer with a mutation in ROS. The invention further provides the use of ganetespib or a pharmaceutically acceptable salt thereof for the manufacture of a medicament for the treatment of a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a cancer with a mutation in ROS. The invention further provides the use of compound of 1A or a pharmaceutically acceptable salt thereof for the manufacture of a medicament for the treatment of a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a NSCLC with a mutation in ROS. The invention further provides the use of compound of 1A or a pharmaceutically acceptable salt thereof for the manufacture of a medicament for the treatment of a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a glioblastoma with a mutation in ROS. The invention further provides the use of compound of 1A or a pharmaceutically acceptable salt thereof for the manufacture of a medicament for the treatment of a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a head and neck cancer, colon cancer, breast cancer, liver cancer, stomach cancer, or kidney cancer with a mutation in ROS. The invention further provides the use of compound of 1A or a pharmaceutically acceptable salt thereof for the manufacture of a medicament for the treatment of a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a cancer with a mutation in ROS. The invention also provides a compound of structural formula (I) or (Ia) or a compound in Tables 1 or 2 or a pharmaceutically acceptable salt thereof for use in treating a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a NSCLC with a mutation in ROS. The invention also provides a compound of structural formula (I) or (Ia) or a compound in Tables 1 or 2 or a pharmaceutically acceptable salt thereof for use in treating a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a glioblastoma with a mutation in ROS. The invention also provides a compound of structural formula (I) or (Ia) or a compound in Tables 1 or 2 or a pharmaceutically acceptable salt thereof for use in treating a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a head and neck cancer, colon cancer, breast cancer, liver cancer, stomach cancer, or kidney cancer with a mutation in ROS. The invention also provides a compound of structural formula (I) or (Ia) or a compound in Tables 1 or 2 or a pharmaceutically acceptable salt thereof for use in treating a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a cancer with a mutation in ROS. The invention also provides ganetespib or a pharmaceutically acceptable salt thereof for use in treating a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a NSCLC with a mutation in ROS. The invention also provides ganetespib or a pharmaceutically acceptable salt thereof for use in treating a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a glioblastoma with a mutation in ROS. The invention also provides ganetespib or a pharmaceutically acceptable salt thereof for use in treating a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a head and neck cancer, colon cancer, breast cancer, liver cancer, stomach cancer, or kidney cancer with a mutation in ROS. The invention also provides ganetespib or a pharmaceutically acceptable salt thereof for use in treating a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a cancer with a mutation in ROS. The invention also provides compound of 1A or a pharmaceutically acceptable salt thereof for use in treating a subject with a cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a NSCLC with a mutation in ROS. The invention also provides compound of 1A or a pharmaceutically acceptable salt thereof for use in treating a subject with a NSCLC with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a glioblastoma with a mutation in ROS. The invention also provides compound of 1A or a pharmaceutically acceptable salt thereof for use in treating a subject with a glioblastoma with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a head and neck cancer, colon cancer, breast cancer, liver cancer, stomach cancer, or kidney cancer with a mutation in ROS. The invention also provides compound of 1A or a pharmaceutically acceptable salt thereof for use in treating a subject with a head and neck cancer, colon cancer, breast cancer, liver cancer, stomach cancer, or kidney cancer with a mutation in ROS in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of a compound of structural formula (I) or (Ia) or a compound in Tables 1 or 2 or a pharmaceutically acceptable salt thereof for the manufacture of a medicament for the treatment of a subject with a cancer with a mutation in RET. The invention further provides the use of a compound of structural formula (I) or (Ia) or a compound in Tables 1 or 2 or a pharmaceutically acceptable salt thereof for the manufacture of a medicament for the treatment of a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of a compound of structural formula (I) or (Ia) or a compound in Tables 1 or 2 or a pharmaceutically acceptable salt thereof for the manufacture of a medicament for the treatment of a subject with a NSCLC, thyroid cancer, or lung adenocarcinoma with a mutation in RET. The invention further provides the use of a compound of structural formula (I) or (Ia) or a compound in Tables 1 or 2 or a pharmaceutically acceptable salt thereof for the manufacture of a medicament for the treatment of a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a cancer with a mutation in RET. The invention further provides the use of ganetespib or a pharmaceutically acceptable salt thereof for the manufacture of a medicament for the treatment of a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of ganetespib or a pharmaceutically acceptable salt thereof for the manufacture of a medicament for the treatment of a subject with a NSCLC, thyroid cancer, or lung adenocarcinoma with a mutation in RET. The invention further provides the use of ganetespib or a pharmaceutically acceptable salt thereof for the manufacture of a medicament for the treatment of a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a cancer with a mutation in RET. The invention further provides the use of compound of 1A or a pharmaceutically acceptable salt thereof for the manufacture of a medicament for the treatment of a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides the use of compound of 1A or a pharmaceutically acceptable salt thereof for the manufacture of a medicament for the treatment of a subject with a NSCLC, thyroid cancer, or lung adenocarcinoma with a mutation in RET. The invention further provides the use of compound of 1A or a pharmaceutically acceptable salt thereof for the manufacture of a medicament for the treatment of a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a cancer with a mutation in RET. The invention also provides a compound of structural formula (I) or (Ia) or a compound in Tables 1 or 2 or a pharmaceutically acceptable salt thereof for use in treating a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides a compound of structural formula (I) or (Ia) or a compound in Tables 1 or 2 or a pharmaceutically acceptable salt thereof for use in treating a subject with a NSCLC, thyroid cancer, or lung adenocarcinoma with a mutation in RET. The invention also provides a compound of structural formula (I) or (Ia) or a compound in Tables 1 or 2 or a pharmaceutically acceptable salt thereof for use in treating a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a cancer with a mutation in RET. The invention also provides ganetespib or a pharmaceutically acceptable salt thereof for use in treating a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides ganetespib or a pharmaceutically acceptable salt thereof for use in treating a subject with a NSCLC, thyroid cancer, or lung adenocarcinoma with a mutation in RET. The invention also provides ganetespib or a pharmaceutically acceptable salt thereof for use in treating a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a cancer with a mutation in RET. The invention also provides compound of 1A or a pharmaceutically acceptable salt thereof for use in treating a subject with a cancer with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention also provides compound of 1A or a pharmaceutically acceptable salt thereof for use in treating a subject with a NSCLC, thyroid cancer, or lung adenocarcinoma with a mutation in RET. The invention also provides compound of 1A or a pharmaceutically acceptable salt thereof for use in treating a subject with a NSCLC, thyroid cancer, or lung adenocarcinoma with a mutation in RET in combination with one or more of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

The invention is illustrated by the following examples, which are not intended to be limiting in any way.

### Examples

### A. Materials and Methods

Cell Lines

Human HCC78 NSCLC cells and human TPC-1 thyroid cancer cells were obtained from Dr. Steve Morris (St. Jude's) and grown in RPMI in the presence of fetal bovine serum (10%), 2 mM L-glutamine and antibiotics (100 IU/ml penicillin and 100 μg/ml streptomycin) purchased from Sigma Aldrich. Cells were maintained at 37° C., 5% CO₂ atmosphere.

Western Blotting

Cells, treated with compound for 24 hr, were lysed in RIPA buffer (CST, Danvers, Mass., USA) on ice and clarified by centrifugation. Equal amounts of proteins were resolved by SDS-PAGE and immunoblotted with indicated antibodies (CST). The antigen-antibody complex was visualized and quantitated using an Odyssey system (LI-COR, Lincoln, Nebr., USA).

Cell Viability Assays

Cell viability was measured using the Cell Titer-Glo assay (Promega). In brief, cells were plated in 96-well plates in triplicate at optimal seeding density (determined empirically for each cell line) and incubated at 37° C., 5% CO₂ atmosphere for 24 hr prior to the addition of drug or vehicle (0.3% DMSO) to the culture medium. At the end of the assay, Cell Titer-Glow was added to the wells per manufactures recommendation, shaken for two minutes and incubated for 10 minutes at room temperature. Luminescence (0.1 sec) was measured with a Victor II microplate reader (Perkin Elmer) and the resulting data were used to calculate cell viability, normalized to vehicle control.

### B. Ganetespib Modulates RET and ROS Fusion Kinase Activity

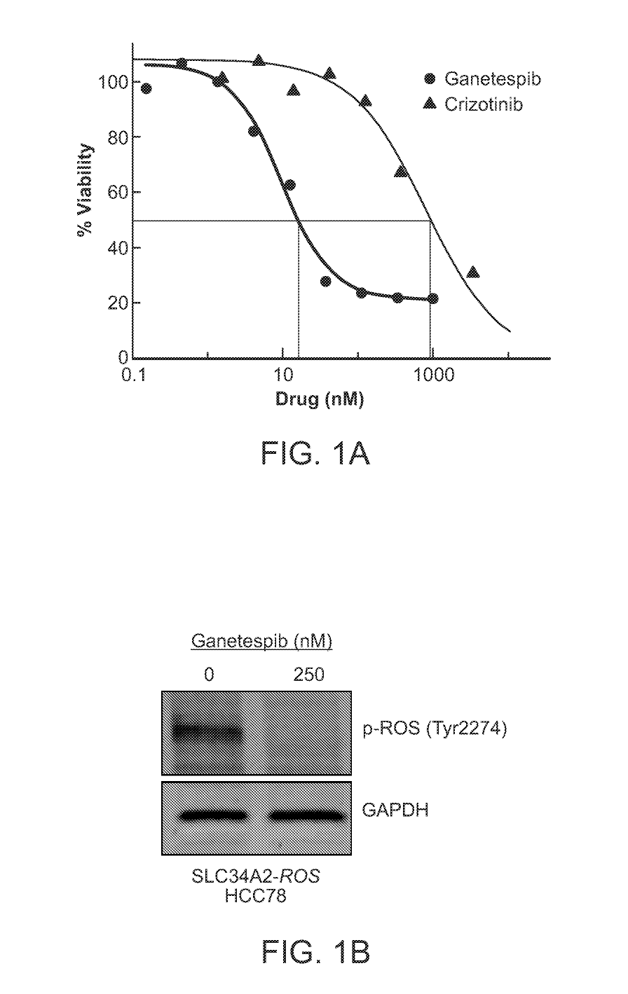

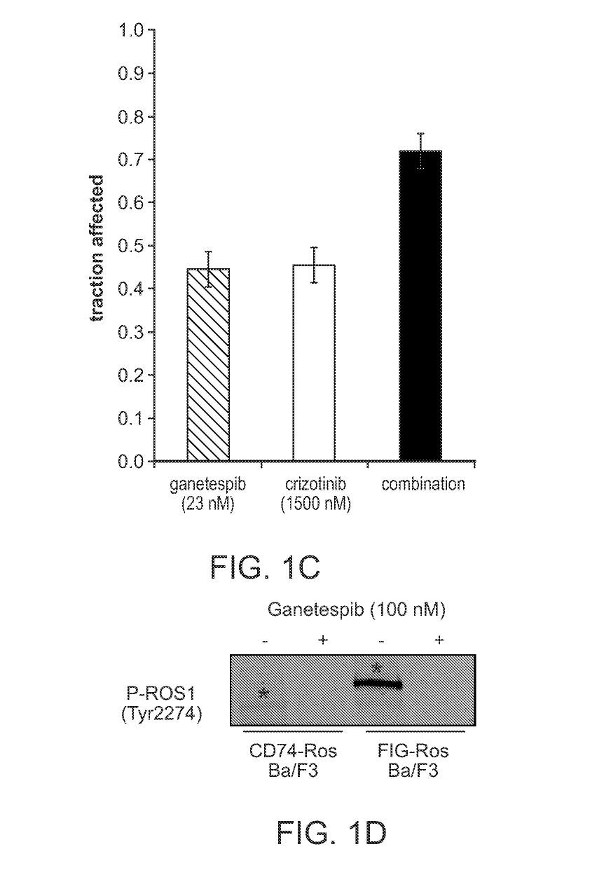

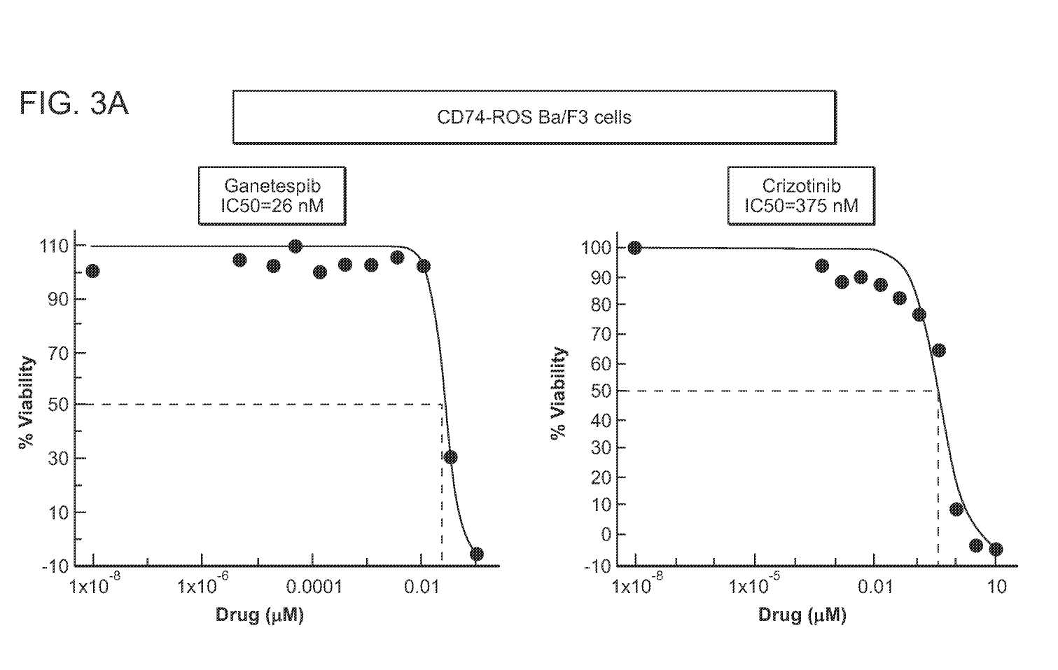

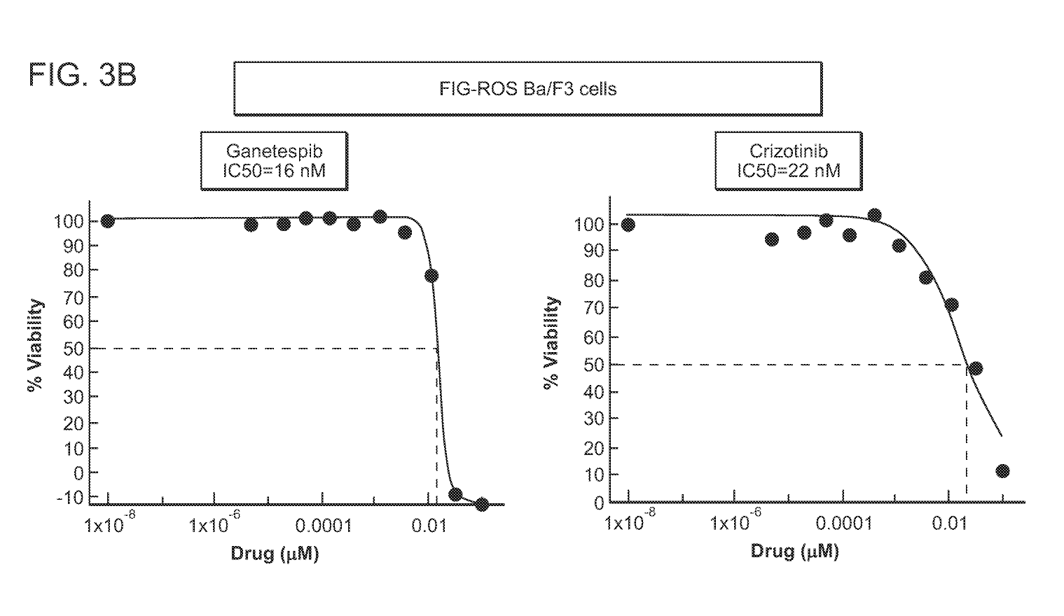

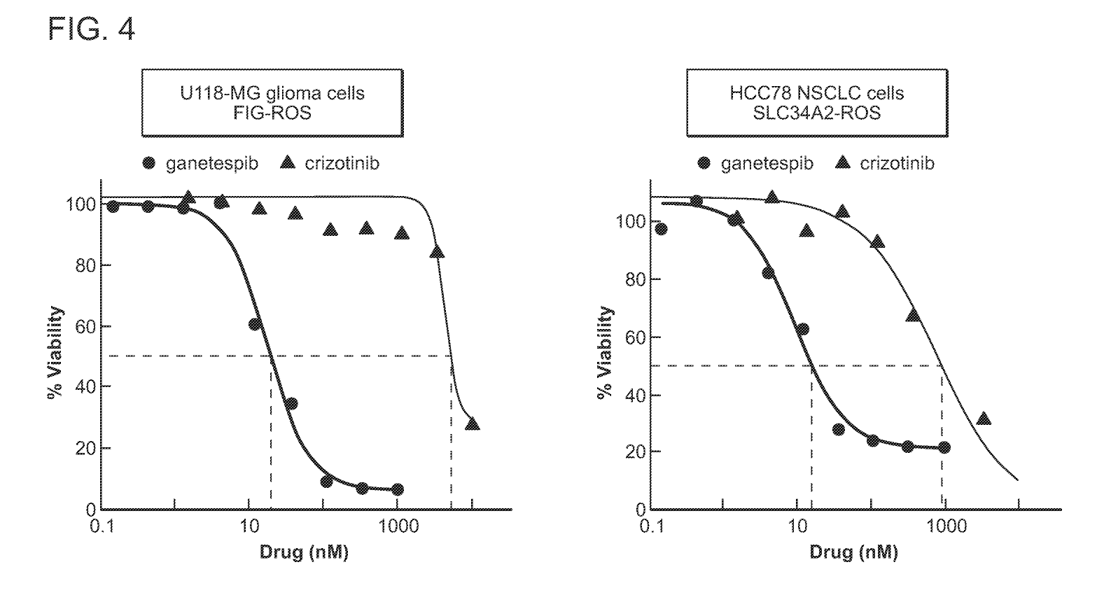

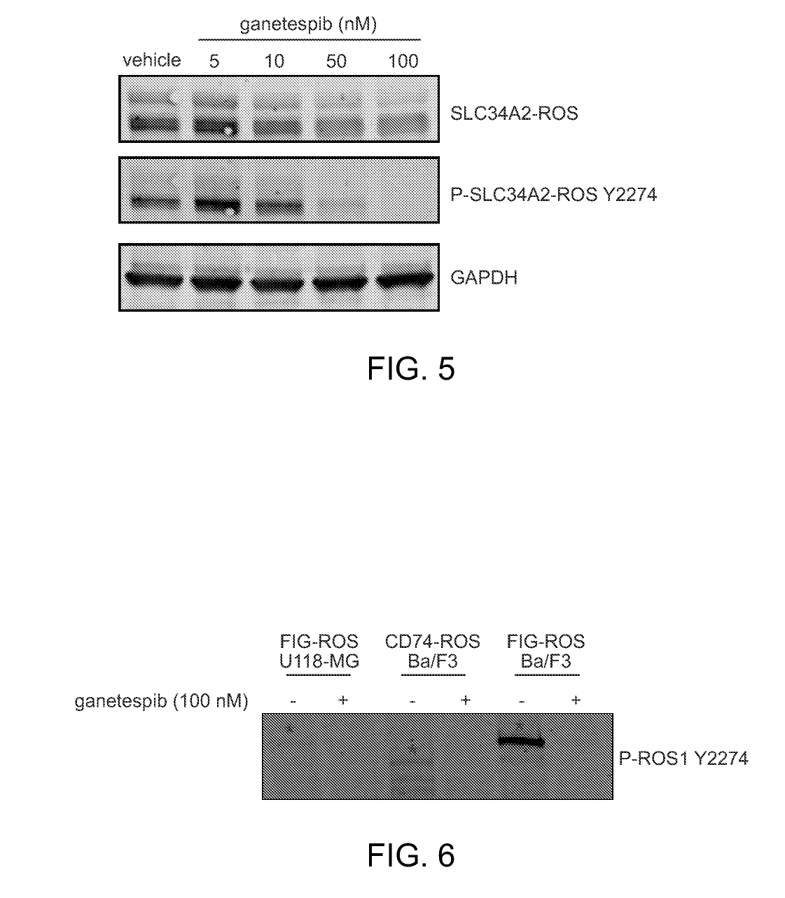

Chromosomal rearrangements involving ROS kinase have been reported in several tumor types, including non-small cell lung cancer (NSCLC) and glioblastoma. Such rearrangements have been reported to be transformative on their own in preclinical studies suggesting that inhibition of ROS activity may provide a means to treat patients with cancers that expresses ROS fusions. See, e.g., Ting-Lei Gu et al, PLoS One. 2011; 6(1):e15640. To determine if inhibition of Hsp90 affects the activity of ROS fusion kinases and viability of cells driven by such fusions, SLC34A2-ROS expressing HCC78 NSCLC cells were treated with ganetespib and both viability and protein activity was assessed. Shown in FIG. 1A, ganetespib displays potent anticancer activity in HCC78 cells (IC50=17 nM). The MET/ALK inhibitor crizotinib has been shown to kill HCC78 cells because of its ability to inhibit ROS. See, e.g., Chenguang Li et al, PLoS One. 2011; 6(11):e28204. While weaker than ganetespib, crizotinib treatment resulted in the loss of HCC78 cell viability (IC50=1035 nM). To determine if ganetespib affects the constitutive activity of ROS, HCC78 cells were treated with ganetespib for 24 hr and the phosphorylation of ROS was assayed by Western blot. Shown in FIG. 1B, ganetespib treatment resulted in significant dephosphorylation of ROS. Given that ganetespib and crizotinib alter ROS through different mechanisms, combinations of the two drugs were investigated to determine if dual blockade of ROS would be more effective than monotherapy. Shown in FIG. 1C, doses of ganetespib and crizotinib that kill approximately 50% of the cells on their own resulted in 70% cell death when combined together. In addition to SLC34A2-ROS, other ROS chimeras have been identified including CD74-ROS and FIG-ROS. Show in FIG. 1D, Ba/F3 cells stably expressing either CD74-ROS or FIG-ROS were treated with ganetespib for 24 hr and activity of ROS was determined by Western blot. From the data, ganetespib effectively reduces the activity of both fusion kinases. FIGS. 3-6 show even more data on ganetespib in reducing the activity of ROS fusion protein.

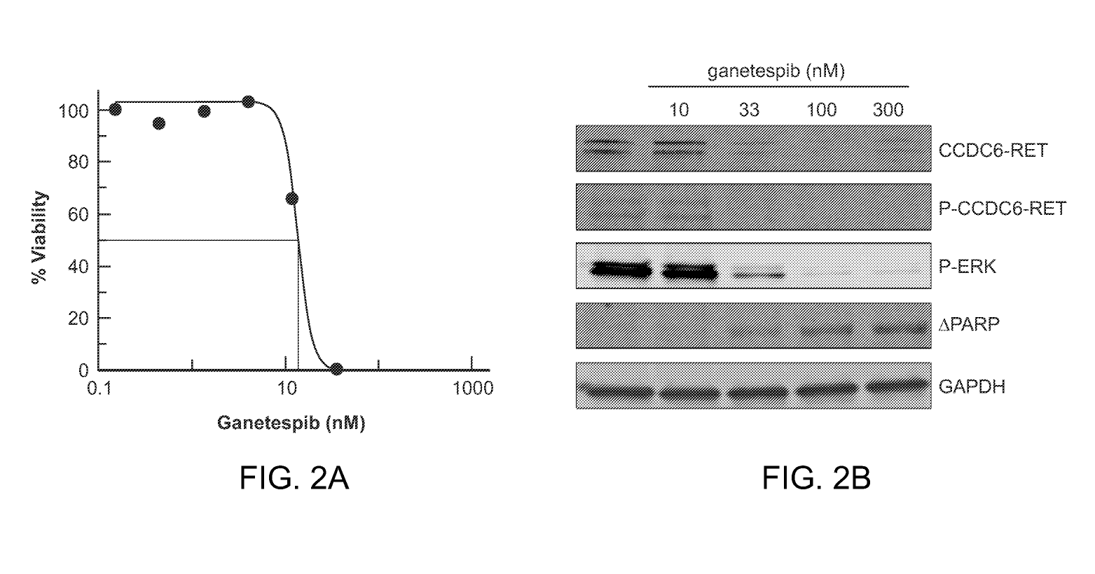

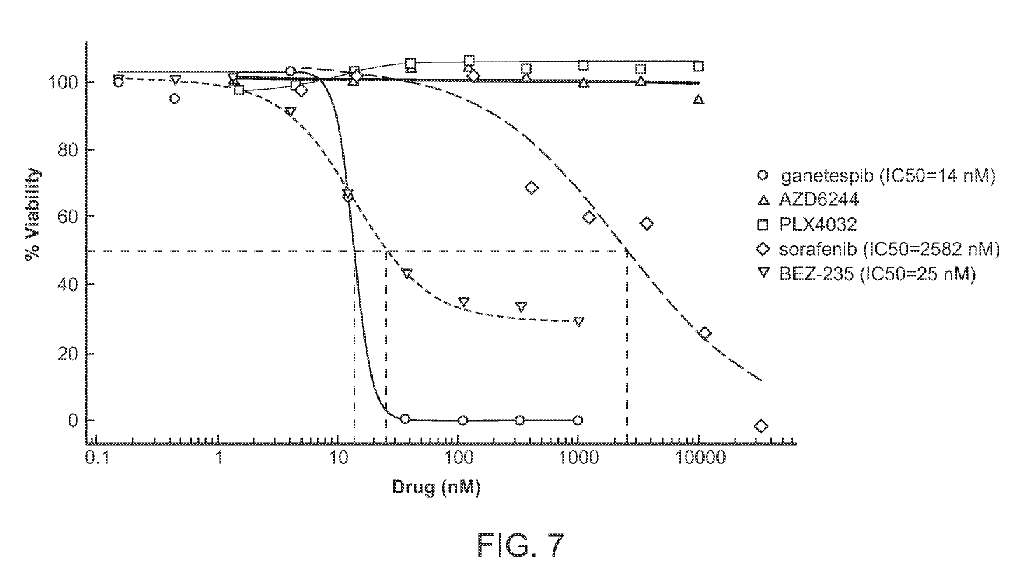

Recently, fusions of the RET kinase have also been detected in NSCLC. See, e.g., Fei Li et al, Cell Research (2012), 1-4. One such fusion, CCDC6-RET, was determined to be present in ˜1% of lung adenocarcinomas from never smokers. To investigate whether ganetespib modulates the activity and stability of RET fusions, CCDC6-RET expressing TPC-1 thyroid cancer cells were used as a model. Shown in FIG. 2, ganetespib potently induced the degradation of total and phosphorylated CCDC6-RET, deactivation of ERK and cleavage of the pro-apoptotic protein PARP, resulting in cell death. FIG. 7 shows further data about ganetespib in modulating the activity of RET fusion protein.

In summary, ganetespib is effective in treating cancer with a ROS or RET mutation either as a single agent or in combination with crizotinib.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A method of treating cancer or tumor in a subject with a mutation in ROS, comprising the steps of:
a) identifying a subject with cancer with a mutation in ROS; and
b) administering to the subject an effective amount of an Hsp90 inhibitor according to the following formulae: 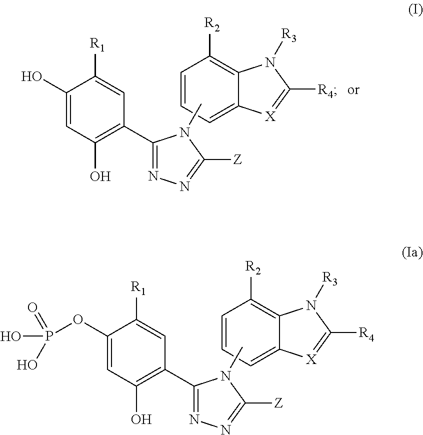
or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈NR₁₀R₁₁, —SC(S)NR₁₀R₁₁, —OC(NR₅)R₇, —SC(NR₅)R₇, —C(O)NR₁₀R₁₁, —NR₅C(O)R₇, —NR₇C(S)R₇, —NR₇C(S)OR₇, —NR₇C(NR₅)R₇, —NR₇C(O)OR₇, —NR₇C(NR₅)OR₇, —NR₇C(O)NR₁₀R₁₁, —NR₇C(S)NR₁₀R₁₁, —NR₇C(NR₈)R₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₅S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₅)R₇, —SC(NR₅)R₇, —NR₇C(NR₅)R₇, —OC(NR₅)OR₇, —SC(NR₅)OR₇, —NR₇C(NR₅)OR₇, —OC(NR₈)NR₁₀R₁₁, —SC(NR₈)NR₁₀R₁₁, or —NR₇C(NR₅)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

2. The method of claim 1, wherein the Hsp90 inhibitor is selected from the group consisting of:
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
and
3-(2,4-dihydroxy-5-isopropyl-phenyl)-4-(1-propyl-indol-5-yl)-5-mercapto-[1,2,4]triazole,
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate,
or a tautomer, or a pharmaceutically acceptable salt thereof.

3. The method of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

4. The method of claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

5. The method of claim 1, wherein the cancer with a mutation in ROS is non-small cell lung cancer or glioblastoma.

6. The method of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof; and the cancer with a mutation in ROS is non-small cell lung cancer or glioblastoma.

7. The method of claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof; and the cancer with a mutation in ROS is non-small cell lung cancer or glioblastoma

8. The method of claim 6, wherein the Hsp90 inhibitor is administered in combination with one or more additional therapeutic agents.

9. The method of claim 8, wherein the one or more therapeutic agents is selected from the group consisting of BEZ235, AZD6244, AZD8055, SN-38, gemcitabine, camptothecin, docetaxel, cisplatin, oxaliplatin, crizotinib, paclitaxel, trastuzumab, pemetrexed, erlotinib, bevacizumab, carboplatin, Abraxane®, bortezomib, topotecan, cetuximab, gemcitabine, and tetracycline.

10. The method of claim 9, wherein the one or more therapeutic agents is erlotinib, crizotinib or bevacizumab.

11. The method of claim 9, wherein the one or more therapeutic agents is docetaxel, paclitaxel or Abraxane®.

12. The method of claim 1, wherein the subject is human.

13. A method of treating cancer in a subject, comprising administering a compound of formulae (I) or (Ia) or a pharmaceutically acceptable salt or tautomer thereof as defined in claim 1, wherein the subject has a cancer with a mutation in ROS.

14. The method of claim 13, wherein the compound is 3-(2,4-dihydroxy-5-isopropyl-phenyl)-4-(1-methyl-indol-5-yl)-5-hydroxy-[1,2,4]triazole.

15. The method of claim 14, wherein the compound is administered in combination with one or more additional therapeutic agents.

16. The method of claim 15, wherein the additional therapeutic agent is crizotinib.

17. The method of claim 14, wherein the cancer is non-small cell lung cancer, glioblastoma, head and neck cancer, colon cancer, breast cancer, liver cancer, stomach cancer, or kidney cancer.

18. A method of treating cancer in a subject, comprising administering a compound of formulae (I) or (Ia) or a pharmaceutically acceptable salt or tautomer thereof as defined in claim 1, wherein the subject has a cancer with a mutation in RET.

19. The method of claim 18, wherein the compound is 3-(2,4-dihydroxy-5-isopropyl-phenyl)-4-(1-methyl-indol-5-yl)-5-hydroxy-[1,2,4]triazole.

20. The method of claim 19, wherein the compound is administered in combination with one or more additional therapeutic agents.

21. The method of claim 20, wherein the additional therapeutic agent is crizotinib.

22. The method of claim 19, wherein the cancer is non-small cell lung cancer, thyroid cancer, or lung adenocarcinoma.
